(19) United States
(12) Patent Application Publication
(10) Pub. No.: US 2026/0256068 A1
(43) Pub. Date: Sep. 3, 2026

(54) ARTIFICIAL POLLINATION SYSTEM, DEVICES, AND COMPONENTS THEREOF

(71) Applicant: BLOOMX LTD, Rishpon (IL)

(72) Inventors: Avi KEREN, Tel Aviv (IL); Thai SADE; Ido-Ad SENESH, Geva Carmel (IL)

(21) Appl. No.: 19/489,953

(22) PCT Filed: Jun. 6, 2024
(86) PCT No.: PCT/IL2024/050565

§ 371 Date: Dec. 4, 2025

Related U.S. Application Data
(60) Provisional application No. 63/471,609, filed on Jun. 7, 2023.

(51) Int. Cl. A01H 1/02

(52) U.S. Cl. CPC A01H 1/027

(57) ABSTRACT

Some embodiments relate to a portable handheld and vehicle-drawn pollination devices configured to attract pollen, comprising a control unit configured for electrically connecting thereto a power source, a pollen attracting component electrically coupled to the control unit and configured to generate an electrostatic field by virtue of high voltage provided by the power source to attract the pollen. Handheld devices include an insulating arrangement insulating at least the pollen attracting component and the control unit from the environment. Vehicle-drawn pollination devices include arms which are passively and/or actively controlled to modulate the forces of contacts with plants, optionally including sensing of forces, and distinguishing of forces specifically due to plant contacts. Some vehicle-drawn devices include matrix transports for the pollen adsorbing matrix which receives the attracted pollen. Some examples include a user interface which provides operator guidance according to sensed device motions and their classification.

### CROSS-REFERENCE TO RELATED APPLICATIONS

This application is a national phase filing under 35 C.F.R. § 371 of and claims priority to PCT Patent Application No. PCT/IL2024/050565, filed on Jun. 6, 2024, which claims the benefit of priority of U.S. Provisional Patent Application No. 63/471,609, filed on Jun. 7, 2023.

The contents of each of the above references are incorporated herein by reference in their entirety. The contents of this reference are incorporated herein by reference in their entirety.

### FIELD AND BACKGROUND OF THE DISCLOSURE

The presently disclosed subject matter, in some embodiments thereof, relates to the field of artificial pollination and more particularly, but not exclusively, to systems, devices, and methods for performing artificial pollination.

Pollination involves transferring pollen grains from the male part of a plant to the female part of a plant (stigma). Natural pollinators include wind, small birds, and insects (especially bees). Globalization may shift crop production into regions where original natural pollinators are rare, absent, have changed behavior, and/or changed efficacy of pollination. Artificial pollination may act to counteract such issues, e.g., by increasing crop yields and/or quality.

### SUMMARY OF THE DISCLOSURE

According to an aspect of some embodiments of the present disclosure, there is provided a pollination system mountable on a carrier configured to carry the system along a pollination path alongside plants to enable the system to assist in pollination of the plants, the system comprising: a support structure; at least one mechanical arm, attached to the support structure; and a pollen exchanger, attached to the arm at a position which engages in contacts with the plants as the system moves along the pollination path; wherein the pollen exchanger comprises a matrix transport which receives a pollen adsorption matrix; and wherein the matrix transport operates to transport portions of the pollen adsorption matrix across the pollen exchanger to refresh a plant-engaging surface defined on a side of the pollen exchanger facing forward along the pollination path.

According to some embodiments of the present disclosure, the at least one mechanical arm comprises a plurality of mechanical arms, arranged to place their respective pollen exchangers at different respective heights.

According to some embodiments of the present disclosure, the pollen adsorption matrix is in sheet form.

According to some embodiments of the present disclosure, the matrix transport holds the pollen adsorption matrix sheet in rolled form.

According to some embodiments of the present disclosure, the matrix transport winds the pollen adsorption matrix from a first roller to a second roller, thereby refreshing the plant-engaging surface.

According to some embodiments of the present disclosure, the matrix transport holds the pollen adsorption matrix sheet in a folded form.

According to some embodiments of the present disclosure, the matrix transport holds the pollen adsorption matrix sheet in the form of a loop.

According to some embodiments of the present disclosure, the matrix transport comprises at least one motor, operable to advance the pollen adsorption matrix sheet and refresh the plant-engaging surface.

According to some embodiments of the present disclosure, the matrix transport operates to transport the pollen adsorption matrix in either of two directions.

According to some embodiments of the present disclosure, there is provided a controller commands the matrix transport to transport the pollen adsorption matrix based on progress of a current operation to collect pollen from the plants and/or pollinate the plants.

According to some embodiments of the present disclosure, there is provided controller having a memory and processor, wherein the memory stores instructions which instruct the processor to: access an indication of progress of a current operation to collect pollen from the plants and/or pollinate the plants; determine, using the indication, that the plant-engaging surface of the pollen exchange should be at least partially refreshed; and initiate at least partial refreshing of the plant-engaging surface, by instructing the transport to transport a portion of the pollen adsorption matrix across the pollen exchanger.

According to some embodiments of the present disclosure, the indication is an indication that the pollination system has advanced alongside a new plant.

According to some embodiments of the present disclosure, the indication is an indication of a distance traveled by the pollination system.

According to some embodiments of the present disclosure, the indication is an indication of a duration of operation of the pollination system.

According to some embodiments of the present disclosure, the matrix transport is configured to transport the pollen adsorption matrix during contacts with a particular one of the plants.

According to some embodiments of the present disclosure, the matrix transport is configured to transport the pollen adsorption matrix in two different directions during the contacts with the particular one of the plants.

According to some embodiments of the present disclosure, the pollen adsorption matrix comprise a dielectric material.

According to some embodiments of the present disclosure, the dielectric material comprises a woven polymer.

According to some embodiments of the present disclosure, the woven polymer is woven to a yarn density of at least 300 Denier.

According to some embodiments of the present disclosure, the woven polymer is woven to a yarn density of at least 420 Denier.

According to some embodiments of the present disclosure, the pollen exchanger comprises an electrically powered pollen attractor comprising an insulated electrode which generates an electrostatic field upon receiving high voltage.

According to some embodiments of the present disclosure, the matrix transport draws the pollen adsorption matrix across the pollen attractor to refresh the plant-engaging surface.

According to some embodiments of the present disclosure, the pollination system includes a size-selective barrier extending across the plant-engaging surface, and including apertures sized to restrict which sizes of plant parts are able to contact the pollen adsorption matrix.

According to some embodiments of the present disclosure, the size-selective barrier comprises a net.

According to some embodiments of the present disclosure, the size-selective barrier includes barrier strands defining a plurality of spacing distances between adjacent the barrier strands.

According to some embodiments of the present disclosure, the plurality of spacing distances are selected to induce vibrations in plants contacting the size-selective barrier at a plurality of frequencies as the carrier moves along the pollination path.

According to some embodiments of the present disclosure, apertures of the size-selective barrier are sized to admit plant flowers, while excluding plant foliage.

According to some embodiments of the present disclosure, apertures of the size-selective barrier are sized to admit pollen, while excluding other plant parts.

According to some embodiments of the present disclosure, the matrix transport operates as the system moves along the pollination path.

According to an aspect of some embodiments of the present disclosure, there is provided a pollination system mountable on a carrier configured to carry the system along a pollination path alongside plants to enable the system to assist in pollination of the plants, the system comprising: a support structure; at least one mechanical arm, attached to the support structure, and positioning a plant-engaging surface of a pollen exchanger where the plant-engaging surface engages in contacts with the plants as the system moves along the pollination path; a motor, attached to the at least one mechanical arm, and operable to change at least an angle at which the plant-engaging surface contacts the plants; and a controller, configured to operate the motor to adjust the angle in response to one or more indications of force exerted upon the plants by the contacts.

According to some embodiments of the present disclosure, the one or more indications of force include a sensed indication of a change in the angle produced by the contacts with the plants.

According to some embodiments of the present disclosure, the one or more indications of force include an indication of a change in how force exerted through the motor affects the angle produced by the contacts with the plants.

According to some embodiments of the present disclosure, the controller operates the motor to reduce force of plant contact in response to the one or more indications of force.

According to some embodiments of the present disclosure, the controller responds to indications of force of plant contact at least greater than 15 N by movement of the mechanical arm to an increased raking angle.

According to some embodiments of the present disclosure, the controller responds to indications of force of plant contact at least greater than 4 N by movement of the mechanical arm to an increased raking angle.

According to some embodiments of the present disclosure, the controller initiates the movement to the increased raking angle by powering the motor to refuse the arm in a direction opposite a direction of travel of the system along the pollination path.

According to some embodiments of the present disclosure, the controller initiates the movement to the increased raking angle by reducing power to the motor, allowing the arm to swing in a direction opposite a direction of travel of the system along the pollination path.

According to some embodiments of the present disclosure, the one or more indications of force combine indications of force exerted on the mechanical arm by plants, and indications of force exerted on the mechanical arm from one or more additional sources.

According to some embodiments of the present disclosure, the one or more additional sources comprise at least one of a tilt angle of the system and an inertial state of the system, with measurements thereof provided among the indications of force.

According to some embodiments of the present disclosure, the controller estimates force exerted on the mechanical arm by plants by subtracting estimated force exerted on the mechanical arm by the one or more additional sources from a total force estimated from the one or more indications of force.

According to some embodiments of the present disclosure, the controller comprises a memory and processor, wherein the memory stores instructions which instruct the processor to: access sensor inputs indicative of forces exerted on the at least one mechanical arm; estimate a force exerted on the at least one mechanical arm by plants, using the sensor inputs; and initiate operation of the motor to adjust the angle of the arm, according to the estimated force.

According to some embodiments of the present disclosure, the controller initiates operation of the motor to decrease the estimated force exerted on the mechanical arm by plants, according to a comparison of the estimated force with a threshold force.

According to some embodiments of the present disclosure, the threshold force is less than about 15 N.

According to some embodiments of the present disclosure, the adjusted angle is an angle around an axis of rotation which is substantially vertical.

According to some embodiments of the present disclosure, the adjusted angle is an angle around an axis of rotation which is substantially horizontal.

According to some embodiments of the present disclosure, the pollen exchanger comprises an electrically powered pollen attractor comprising an insulated electrode which generates an electrostatic field upon receiving high voltage.

According to some embodiments of the present disclosure, the pollination system is provided a pollen adsorption matrix extending across the pollen attractor and providing the plant-engaging surface, wherein the electrostatic field attracts pollen to the pollen adsorption matrix.

According to some embodiments of the present disclosure, the pollination system is provided with a size-selective barrier extending across the plant-engaging surface, and positioned to restrict sizes of plant parts which contact the pollen adsorption matrix.

According to some embodiments of the present disclosure, the mechanical arm includes a flexible or jointed region which is biased to hold a portion of the mechanical arm in a more forward position, but bend under a sufficient force to relieve plant contact forces.

According to some embodiments of the present disclosure, the controller operates the motor to adjust the angle to a default angle, unless the one or more indications indicate that this would exert an excessive force upon plant parts contact the at least one mechanical arm.

According to some embodiments of the present disclosure, the default angle is about perpendicular to the pollination path.

According to an aspect of some embodiments of the present disclosure, there is provided a portable handheld pollination device configured to attract pollen, the device comprising: a power controller at a handheld proximal end of the pollination device, operated through at least one user-operated control, and configured to electrically connect a power source thereto; a pollen attractor electrically coupled to the power controller via an interconnecting reach extender, and having a pollen-attracting surface, wherein the pollen attractor is configured to generate an electrostatic field by virtue of high voltage powered by the power source for attracting the pollen; a user interface; and a processor and memory, wherein the memory stores instructions which instruct the processor to: access one or more indications of operation of the pollination device, evaluate the indications according to one or more criteria of targeted device operation, and operate the user interface to provide one or more indications providing guidance regarding operation of the pollination device, based on the evaluation.

According to some embodiments of the present disclosure, the device comprises one or more motion sensors; and the memory stores instructions which instruct the processor to: access indications of motion of the pollination device provided by the one or more motion sensors, classify the motion, and operate the user interface to provide one or more indications providing guidance regarding operation of the pollination device.

According to some embodiments of the present disclosure, the motion is classified according to height locations reached by the pollen attractor, and the provided guidance is indicative of heights reached during a recent period of operation of the pollination device.

According to some embodiments of the present disclosure, the motion is classified according to a pattern of brushing motions performed with the pollen attractor, and the provided guidance is indicative of brushing motions performed during recent period of operation of the pollination device.

According to some embodiments of the present disclosure, the pollen attractor is configured to receive a detachable pollen cartridge providing a pollen adsorption matrix; wherein the motion is classified according to operation of the pollen attractor in an elevated position; and wherein the provided guidance is indicative of a sufficiency of time of operation in an elevated position, since the last replacement of the detachable pollen cartridge.

According to an aspect of some embodiments of the present disclosure, there is provided the portable handheld pollination device wherein the motion is classified according to operation of the pollen attractor in an elevated position; and wherein the provided guidance is indicative of a sufficiency of time of operation in an elevated position, for a plant with which the pollination device is currently engaged.

According to some embodiments of the present disclosure, the one or more indications comprise indications of times of device operations, and the criteria target at least one of: a duration of continuous device operation, a cumulative time of device operation since a selected event, and a period during which the device is targeted to be operated.

According to an aspect of some embodiments of the present disclosure, there is provided a pollination system comprising: a pollen exchanger carrying a pollen adsorption matrix; wherein the pollen exchanger includes a size-selective barrier extending across the pollen adsorption matrix; and the size-selective barrier has apertures sized to restrict which sizes of plant parts are able to contact the pollen adsorption matrix.

According to some embodiments of the present disclosure, the size-selective barrier comprises a net.

According to some embodiments of the present disclosure, the size-selective barrier includes barrier strands defining a plurality of spacing distances between adjacent the barrier strands.

According to some embodiments of the present disclosure, the plurality of spacing distances are selected to induce vibrations in plants contacting the size-selective barrier at a plurality of frequencies as the pollen exchanger moves relative to engaged plant parts.

According to some embodiments of the present disclosure, apertures of the size-selective barrier are sized to admit plant flowers, while excluding plant foliage.

According to some embodiments of the present disclosure, apertures of the size-selective barrier are sized to admit pollen, while excluding other plant parts.

According to an aspect of some embodiments of the present disclosure, there is provided a portable handheld pollination device configured to attract pollen, the device comprising: a handle, sized to be held by an operator; a pollen exchanger; and a reach extender interconnecting the handle and the pollen exchanger; wherein the pollen exchanger comprises a matrix transport which receives a pollen adsorption matrix; and wherein the matrix transport operates to transport portions of the pollen adsorption matrix across the pollen exchanger to refresh a plant-engaging surface defined on a side of the pollen exchanger which exposes the pollen adsorption matrix to plant contacts.

According to some embodiments of the present disclosure, the handle includes at least one operator-activated control, which controls operation of the matrix transport.

According to some embodiments of the present disclosure, the matrix transport operations to induce relative movement of the pollen adsorbing matrix relative to contacted plant parts.

According to an aspect of some embodiments of the present disclosure, there is provided a fabric used as a pollen adsorbing matrix for pollen collection, the fabric comprising a woven polymer of about 300 Denier or coarser, wherein the fabric is mounted on a pollination device with a first side of the fabric facing a pollen attractor configured to generate an electrostatic field of at least 1 kV, and a second side presenting a pollen collecting surface to collect pollen.

According to some embodiments of the present disclosure, the pollination device is also used for pollination.

According to some embodiments of the present disclosure, yarns of the woven polymer comprise five or fewer fibers across their thickness.

According to some embodiments of the present disclosure, the fabric comprises a PVC coating.

According to an aspect of some embodiments of the present disclosure, there is provided a portable handheld pollination device configured to attract pollen, the device comprising: a power controller at a handheld proximal end of the pollination device, operated through at least one user-operated control, and configured to electrically connect a power source thereto; a pollen attractor electrically coupled to the power controller via an interconnecting reach extender, and having a pollen-attracting surface, wherein the pollen attractor is configured to generate an electrostatic field by virtue of high voltage powered by the power source for attracting the pollen; and an insulation arrangement insulating at least the pollen attractor and the power controller, and including insulating along the reach extender, to maintain the electrostatic field and prevent discharge of the high voltage into surroundings of the pollination device.

According to some embodiments of the present disclosure, the pollen attractor comprises an electrode which receives the high voltage to generate the electrostatic field.

According to some embodiments of the present disclosure, the insulation arrangement comprises a first insulating material insulating at least partially the pollen attractor and a second insulating material insulating at least partially the power controller, wherein the first insulating material has a first dielectric strength and the second insulating material has a second dielectric strength lesser than the first dielectric strength.

According to some embodiments of the present disclosure, a coating of the first insulating material has a first thickness and a coating of the second insulating material has a second thickness greater than the first thickness.

According to some embodiments of the present disclosure, the first insulating material is a dielectric material.

According to some embodiments of the present disclosure, the second insulating material is a dielectric material.

According to some embodiments of the present disclosure, the pollination device comprises a handle configured to be held by a user, the insulation arrangement insulating the handle.

According to some embodiments of the present disclosure, the reach extender has a length ranging between 1 m and 6 m.

According to some embodiments of the present disclosure, the pollination device comprises one or more LEDs, the insulation arrangement insulating the one or more LEDs.

According to some embodiments of the present disclosure, the pollination device further comprises one or more solder cables electrically connecting the power controller and the pollen attractor, the insulation arrangement insulating the one or more solder cables.

According to some embodiments of the present disclosure, the pollination device comprises at least one floating grounding protrusion electrically coupled, at least indirectly, to the power controller to enable grounding of excess charge accumulated on the portable handheld pollination device, the floating grounding protrusion having a conductive portion configured to be electrically exposed to surroundings of the pollination device.

According to some embodiments of the present disclosure, the pollination device comprises a proximal portion configured to be held by a user for operating the pollination device and a distal portion spaced apart from the proximal portion and comprising the pollen attractor, wherein the at least one floating grounding protrusion is positioned at the distal portion in proximity to the pollen attractor.

According to some embodiments of the present disclosure, the at least one floating grounding protrusion is formed as a protrusion protruding from a location in proximity to the pollen attractor.

According to some embodiments of the present disclosure, the protrusion is elastically resilient.

According to some embodiments of the present disclosure, the at least one floating grounding protrusion is formed as a frame.

According to some embodiments of the present disclosure, the conductive portion is configured to be electrically disposed to surroundings of the pollination device at least when the pollen attractor attracts the pollen from a plant.

According to some embodiments of the present disclosure, the conductive portion is configured to engage the plant at least when the pollen attractor attracts the pollen from the plant.

According to some embodiments of the present disclosure, the conductive portion is configured to enable grounding of the excess charge accumulated on the portable handheld pollination device through the plant.

According to some embodiments of the present disclosure, the pollination device comprises a detachably attachable pollination cartridge having a pollen adsorbing matrix configured at least to collect the pollen attracted by the pollen attractor.

According to some embodiments of the present disclosure, the pollen adsorbing matrix is configured to independently store the collected pollen at least after the pollination cartridge is detached from the pollination device.

According to some embodiments of the present disclosure, the pollen adsorbing matrix comprises a dielectric material.

According to some embodiments of the present disclosure, the pollen adsorbing matrix is configured to release the collected pollen to parts of flowers receptive to pollination.

According to some embodiments of the present disclosure, the pollen adsorbing matrix is configured for the collected pollen to be wiped off the pollen adsorbing matrix by contacting parts of flowers receptive to pollination.

According to some embodiments of the present disclosure, the pollen adsorbing matrix is elastically resilient.

According to some embodiments of the present disclosure, the pollination cartridge further comprises one or more attachment elements configured for detachably attaching the pollination cartridge to the pollination device.

According to some embodiments of the present disclosure, at least a majority of the one or more attachment elements is disposed at a periphery of the pollination cartridge.

According to some embodiments of the present disclosure, a thickness of the pollen adsorbing matrix ranges between 0.1 mm to 50 mm.

According to some embodiments of the present disclosure, a dielectric strength of the dielectric material ranges between 1 kV/mm to 1000 kV/mm.

According to some embodiments of the present disclosure, the dielectric material is an electrical insulating material.

According to some embodiments of the present disclosure, a ratio between the dielectric strength and the thickness ranges between 0.3 kV/mm² to 800 kV/mm².

According to some embodiments of the present disclosure, the pollination device comprises one or more pollination cartridge attachment elements configured for detachably attaching the pollination cartridge at least partially over the pollen-attracting surface.

According to an aspect of some embodiments of the present disclosure, there is provided a portable handheld pollination device configured to attract pollen, the device comprising: a power controller configured for electrically connecting thereto a power source; a pollen attractor electrically coupled to the power controller and having a pollen-attracting surface configured to generate an electrostatic field by virtue of high voltage powered by the power source for attracting pollen; and at least one floating grounding protrusion electrically coupled, at least indirectly, to the power controller, enabling grounding of excess charge accumulated on the portable handheld pollination device, the floating grounding protrusion having a conductive portion configured to be electrically exposed to surroundings of the pollination device.

According to some embodiments of the present disclosure, the pollination device comprises a proximal portion configured to be held by a user for operating the pollination device and a distal portion spaced apart from the proximal portion and comprising the pollen attractor, wherein the at least one floating grounding protrusion is positioned at the distal portion in proximity to the pollen attractor.

According to some embodiments of the present disclosure, the at least one floating grounding protrusion is formed as a protrusion protruding from a location in proximity to the pollen attractor.

According to some embodiments of the present disclosure, the protrusion is elastically resilient.

According to some embodiments of the present disclosure, the at least one floating grounding protrusion is formed as a frame.

According to some embodiments of the present disclosure, the conductive portion is configured to be electrically disposed to surroundings of the pollination device at least when the pollen attractor attracts the pollen from a plant.

According to some embodiments of the present disclosure, the conductive portion is configured to engage the plant at least when the pollen attractor attracts the pollen from the plant.

According to some embodiments of the present disclosure, the conductive portion is configured to enable grounding of the excess charge accumulated on the portable handheld pollination device through the plant.

According to some embodiments of the present disclosure, the pollination device comprises an insulation arrangement insulating at least the pollen attractor and the power controller from surroundings of the pollination device.

According to some embodiments of the present disclosure, the pollen attractor comprises an electrode configured to generate the electrostatic field.

According to some embodiments of the present disclosure, the insulation arrangement comprises a first insulating material insulating at least partially the pollen attractor and a second insulating material insulating at least partially the power controller, wherein the first insulating material has a first dielectric strength and the second insulating material has a second dielectric strength lesser than the first dielectric strength.

According to some embodiments of the present disclosure, a coating of the first insulating material has a first thickness and a coating of the second insulating material has a second thickness greater than the first thickness.

According to some embodiments of the present disclosure, the first insulating material is a dielectric material.

According to some embodiments of the present disclosure, the second insulating material is a dielectric material.

According to some embodiments of the present disclosure, the pollination device comprises a handle configured to be held by a user, the insulation arrangement insulating the handle.

According to some embodiments of the present disclosure, the pollination device comprises an extender extending between the handle and the pollen attractor, the insulation arrangement insulating the extender.

According to some embodiments of the present disclosure, the extender has a length ranging between 1 cm to 600 cm.

According to some embodiments of the present disclosure, the pollination device comprises one or more LEDs, the insulation arrangement insulating the one or more LEDs.

According to some embodiments of the present disclosure, the pollination device comprises one or more solder cables electrically connecting the power controller and the pollen attractor, the insulation arrangement insulating the one or more solder cables.

According to some embodiments of the present disclosure, the pollination device comprises a detachably attachable pollination cartridge having a pollen adsorbing matrix configured at least to collect the pollen attracted by the pollen attractor.

According to some embodiments of the present disclosure, the pollen adsorbing matrix is configured to independently store the collected pollen at least after the pollination cartridge is detached from the pollination device.

According to some embodiments of the present disclosure, the pollen adsorbing matrix comprises a dielectric material.

According to some embodiments of the present disclosure, the pollen adsorbing matrix is configured to release the collected pollen to parts of flowers receptive to pollination.

According to some embodiments of the present disclosure, the pollen adsorbing matrix is configured for the collected pollen to be wiped off the pollen adsorbing matrix by parts of flowers receptive to pollination.

According to some embodiments of the present disclosure, the pollen adsorbing matrix is elastically resilient.

According to some embodiments of the present disclosure, the pollination cartridge further comprises one or more attachment elements configured for detachably attaching the pollination cartridge to the pollination device.

According to some embodiments of the present disclosure, at least a majority of the one or more attachment elements is disposed at a periphery of the pollination cartridge.

According to some embodiments of the present disclosure, a thickness of the pollen adsorbing matrix ranges between 0.1 mm to 50 mm.

According to some embodiments of the present disclosure, a dielectric strength of the dielectric material ranges between 1 kV/mm to 1000 kV/mm.

According to some embodiments of the present disclosure, the dielectric material is an electrical insulating material.

According to some embodiments of the present disclosure, a ratio between the dielectric strength and the thickness ranges between 0.3 kV/mm² to 800 kV/mm².

According to some embodiments of the present disclosure, the pollination device comprises one or more pollination cartridge attachment elements configured for detachably attaching the pollination cartridge at least partially over the pollen-attracting surface.

According to an aspect of some embodiments of the present disclosure, there is provided a pollination cartridge for use with a pollination device, the pollination cartridge being configured to be detachably attachable to the pollination device and comprising a pollen adsorbing matrix configured to collect pollen from a source plant, the pollen adsorbing matrix being configured to independently store the collected pollen at least after the pollination cartridge is detached from the pollination device.

According to some embodiments of the present disclosure, the pollen adsorbing matrix comprises a dielectric material.

According to some embodiments of the present disclosure, the pollen adsorbing matrix is configured to release the pollen to flower parts receptive to pollination.

According to some embodiments of the present disclosure, the pollen adsorbing matrix is configured for the collected pollen to be wiped off the pollen adsorbing matrix to release the pollen to flower parts receptive to pollination.

According to some embodiments of the present disclosure, the pollen adsorbing matrix is elastically resilient.

According to some embodiments of the present disclosure, the pollination cartridge comprises one or more attachment elements configured for detachably attaching the pollination cartridge to the pollination device.

According to some embodiments of the present disclosure, at least a majority of the one or more attachment elements is disposed at a periphery of the pollination cartridge.

According to some embodiments of the present disclosure, a thickness of the pollen adsorbing matrix ranges between 0.1 mm to 50 mm.

According to some embodiments of the present disclosure, a dielectric strength of the dielectric material ranges between 1 kV/mm to 1000 kV/mm.

According to some embodiments of the present disclosure, the dielectric material is an electrical insulating material.

According to some embodiments of the present disclosure, a ratio between the dielectric strength and the thickness ranges between 0.3 kV/mm² to 800 kV/mm².

According to an aspect of some embodiments of the present disclosure, there is provided a kit for artificial pollination, the kit comprising: at least one pollination device configured for assisting in at least one of collecting and deploying pollen; at least one pollination cartridge configured to be detachably attachable to the at least one pollination device, the at least one pollination cartridge comprising a pollen adsorbing matrix configured to at least one of: collect the pollen, independently store the collected pollen, and deploy the collected and/or stored pollen.

According to some embodiments of the present disclosure, the at least one pollination cartridge comprises the pollination cartridge according to any of the above-described pollination cartridge features.

According to some embodiments of the present disclosure, the at least one pollination device comprises the pollination device according to any of the above described pollination devices.

According to some embodiments of the present disclosure, the at least one pollination device comprises a pollinating device configured for the at least one pollination cartridge to be detachably attached thereto for deploying the pollen.

According to some embodiments of the present disclosure, the at least one pollination device comprises at least two pollination devices, at least one of the at least two pollination devices being a pollen collector device and at least one of the at least two pollination devices being a pollinating device.

According to some embodiments of the present disclosure, the at least one pollination cartridge is configured to be detachably attachable to each of the at least two pollination devices.

According to some embodiments of the present disclosure, the at least one pollination cartridge is configured to be detachably attachable to the at least one pollen collector device for collecting the pollen, and to the at least one pollinating device for deploying the pollen.

According to an aspect of some embodiments of the present disclosure, there is provided a pollination system mountable on a carrier configured to carry the system along a pollination path alongside plants to enable the system to assist in pollination of the plants, the system comprising: a support structure; a power controller configured for electrically connecting thereto a power source; and at least one mechanical arm having an arm proximal end connected to the support structure and an arm distal end configured to be connected with an electrically powered pollen attractor; and an electrical interface connected to the arm distal end and configured for electrically coupling the power controller and the pollen attractor when connected to the arm distal end; wherein at least the arm distal end has a first degree of freedom with respect to the support structure to allow a movement thereof at least in a first direction extending along the pollination path, wherein the first degree of freedom is at least partially constrained.

According to some embodiments of the present disclosure, movement of the arm distal end through the first degree of freedom is powered by a motor.

According to some embodiments of the present disclosure, the first degree of freedom allows the arm distal end to move in the first direction at least between a forward position and a backward position, the forward and backward positions defining at least partially the constraint of the first degree of freedom.

According to some embodiments of the present disclosure, the at least one mechanical arm is configured to be biased for the arm distal end to be at the forward position, wherein the arm distal end is configured to move into the backward position at least when the plant engaging component, when connected to the arm distal end, engages the plants during forward movement of the carrier along the pollination path.

According to some embodiments of the present disclosure, the at least one mechanical arm is connected to the support structure by a hinged connector configured to provide the first degree of freedom.

According to some embodiments of the present disclosure, the at least one mechanical arm is elastically resilient, thereby providing the first degree of freedom.

According to some embodiments of the present disclosure, at least the arm distal end has a second degree of freedom with respect to the support structure to allow a movement thereof at least in a second direction extending perpendicular to the pollination path as well as to the at least one mechanical arm, wherein the second degree of freedom is at least partially constrained.

According to some embodiments of the present disclosure, the second degree of freedom allows the arm distal end to move in the second direction at least between an upward position and a downward position, the upward and downward positions defining at least partially the constraint of the second degree of freedom.

According to some embodiments of the present disclosure, the at least one mechanical arm is connected to the support structure by a hinged connector configured to provide the second degree of freedom.

According to some embodiments of the present disclosure, the at least one mechanical arm is elastically resilient, thereby providing the second degree of freedom.

According to some embodiments of the present disclosure, the at least one mechanical arm at least partially defines a connection path for providing the electrical coupling between the arm distal end and the power controller.

According to some embodiments of the present disclosure, the pollination system comprises an electrically powered pollen attractor connected at the arm distal end and in electrical connection with the at least one interface, the pollen attractor having a pollen-attracting surface configured to generate an electrostatic field by virtue of high voltage powered by the power source for attracting the pollen.

According to some embodiments of the present disclosure, the pollen attractor comprises an electrode configured to generate the electrostatic field.

According to some embodiments of the present disclosure, the pollination system comprises a pollination cartridge connected, at least indirectly, to the arm distal end, the pollination cartridge comprising a pollen adsorbing matrix configured to engage the plants.

According to some embodiments of the present disclosure, the pollen adsorbing matrix is elastically resilient to flex in a direction extending at least partially along the pollination path at least upon engaging the plant.

According to some embodiments of the present disclosure, the pollination cartridge is configured to be detachably attachable, at least indirectly, to the arm distal end.

According to some embodiments of the present disclosure, at least a portion of the pollen adsorbing matrix is reversibly bendable in a direction extending along the pollination path.

According to some embodiments of the present disclosure, the pollen adsorbing matrix is smooth.

According to some embodiments of the present disclosure, the pollen adsorbing matrix comprises a dielectric material.

According to some embodiments of the present disclosure, the dielectric material is an electrical insulating material.

According to some embodiments of the present disclosure, a thickness of the pollen adsorbing matrix ranges between 0.1 mm to 5 mm.

According to some embodiments of the present disclosure, a dielectric strength of the dielectric material ranges between 1 kV/mm to 1000 kV/mm.

According to some embodiments of the present disclosure, a ratio between the dielectric strength and the thickness ranges between 0.3 kV/mm² to 800 kV/mm².

According to some embodiments of the present disclosure, the pollen adsorbing matrix is configured at least to collect the pollen.

According to some embodiments of the present disclosure, the pollen adsorbing matrix is configured to independently store the collected pollen at least after the pollination cartridge is detached from the arm distal end.

According to some embodiments of the present disclosure, the pollen adsorbing matrix is configured to deploy the collected pollen upon demand.

According to some embodiments of the present disclosure, the pollen adsorbing matrix is configured for the collected pollen to be wiped off the pollen adsorbing matrix for deploying the collected pollen.

According to some embodiments of the present disclosure, the pollination cartridge is configured to be detachably attachable to the pollen attractor.

According to some embodiments of the present disclosure, the pollen adsorbing matrix is configured to collect the pollen attracted by the pollen attractor.

According to an aspect of some embodiments of the present disclosure, there is provided a pollination system mountable on a carrier configured to carry the system along a pollination path alongside plants to enable the system to assist in pollination of the plants, the system comprising: a support structure; at least one mechanical arm having an arm proximal end connected to the support structure and an arm distal end opposite; and a pollen attractor electrically couplable to a power source and having a pollen-attracting surface configured to generate an electrostatic field by virtue of high voltage powered by the power source for attracting pollen, the pollen attractor being configured to be detachably attachable to the arm distal end; wherein at least the arm distal end has a first degree of freedom with respect to the support structure to allow a movement thereof at least in a first direction extending along the pollination path, wherein the first degree of freedom is at least partially constrained.

According to some embodiments of the present disclosure, movement of the arm distal end through the first degree of freedom is powered by a motor.

According to some embodiments of the present disclosure, the first degree of freedom allows the arm distal end to move in the first direction at least between a forward position and a backward position, the forward and backward positions defining at least partially the constraint of the first degree of freedom.

According to some embodiments of the present disclosure, the at least one mechanical arm is configured to be biased for the arm distal end to be at the forward position, wherein the arm distal end is configured to move into the backward position at least when the plant engaging component, when connected to the arm distal end, engages the plants during forward movement of the carrier along the pollination path.

According to some embodiments of the present disclosure, the at least one mechanical arm is connected to the support structure by a hinged connector configured to provide the first degree of freedom.

According to some embodiments of the present disclosure, the at least one mechanical arm is elastically resilient, thereby providing the first degree of freedom.

According to some embodiments of the present disclosure, at least the arm distal end has a second degree of freedom with respect to the support structure to allow a movement thereof at least in a second direction extending perpendicular to the pollination path as well as to the at least one mechanical arm, wherein the second degree of freedom is at least partially constrained.

According to some embodiments of the present disclosure, the second degree of freedom allows the arm distal end to move in the second direction at least between an upward position and a downward position, the upward and downward positions defining at least partially the constraint of the second degree of freedom.

According to some embodiments of the present disclosure, the at least one mechanical arm is connected to the support structure by a hinged connector configured to provide the second degree of freedom.

According to some embodiments of the present disclosure, the at least one mechanical arm is elastically resilient, thereby providing the second degree of freedom.

According to some embodiments of the present disclosure, the pollen attractor comprises an electrode configured to generate the electrostatic field.

According to some embodiments of the present disclosure, the pollination system comprises a pollination cartridge configured to be detachably attachable to the pollen attractor, the pollination cartridge comprising a pollen adsorbing matrix configured to engage the plants.

According to some embodiments of the present disclosure, the pollen adsorbing matrix is elastically resilient to flex in a direction extending at least partially along the pollination path at least upon engaging the plant.

According to some embodiments of the present disclosure, at least a portion of the pollen adsorbing matrix is reversibly bendable in a direction extending along the pollination path.

According to some embodiments of the present disclosure, the pollen adsorbing matrix is smooth.

According to some embodiments of the present disclosure, the pollen adsorbing matrix comprises a dielectric material.

According to some embodiments of the present disclosure, the dielectric material is an electrical insulating material.

According to some embodiments of the present disclosure, a thickness of the pollen adsorbing matrix ranges between 0.1 mm to 5 mm.

According to some embodiments of the present disclosure, a dielectric strength of the dielectric material ranges between 1 kV/mm to 1000 kV/mm.

According to some embodiments of the present disclosure, a ratio between the dielectric strength and the thickness ranges between 0.3 kV/mm² to 800 kV/mm².

According to some embodiments of the present disclosure, the pollen adsorbing matrix is configured at least to collect the pollen attracted by the pollen attractor.

According to some embodiments of the present disclosure, the pollen adsorbing matrix is configured to independently store the collected pollen at least after the pollination cartridge is detached from the arm distal end.

According to some embodiments of the present disclosure, the pollen adsorbing matrix is configured to deploy the collected pollen upon demand.

According to an aspect of some embodiments of the present disclosure, there is provided a pollination system mountable on a carrier configured to carry the system along a pollination path alongside plants to enable the system to assist in pollination of the plants, the system comprising: a support structure; and a mechanical arm having an arm proximal end connected to the support structure and an arm distal end; and a pollination cartridge connected, at least indirectly, to the arm distal end, the pollination cartridge comprising a pollen adsorbing matrix configured to engage the plants, the pollen adsorbing matrix being elastically resilient to flex in a direction extending at least partially along the pollination path at least upon engaging the plant.

According to some embodiments of the present disclosure, the pollination cartridge is configured to be detachably attachable, at least indirectly, to the arm distal end.

According to some embodiments of the present disclosure, at least a portion of the pollen adsorbing matrix is reversibly bendable in a direction extending along the pollination path.

According to some embodiments of the present disclosure, the pollen adsorbing matrix is smooth.

According to some embodiments of the present disclosure, the pollen adsorbing matrix comprises a dielectric material.

According to some embodiments of the present disclosure, the dielectric material is an electrical insulating material.

According to some embodiments of the present disclosure, a thickness of the pollen adsorbing matrix ranges between 0.1 mm to 5 mm.

According to some embodiments of the present disclosure, a dielectric strength of the dielectric material ranges between 1 kV/mm to 1000 kV/mm.

According to some embodiments of the present disclosure, a ratio between the dielectric strength and the thickness ranges between 0.3 kV/mm² to 800 kV/mm².

According to some embodiments of the present disclosure, the pollen adsorbing matrix is configured at least to collect pollen.

According to some embodiments of the present disclosure, the pollen adsorbing matrix is configured to independently store the collected pollen at least after the pollination cartridge is detached from the arm distal end.

According to some embodiments of the present disclosure, the pollen adsorbing matrix is configured to deploy the collected pollen upon demand.

According to some embodiments of the present disclosure, the pollen adsorbing matrix is configured for the collected pollen to be wiped off the pollen adsorbing matrix for deploying the collected pollen.

According to some embodiments of the present disclosure, the pollination system comprises a power controller configured for electrically connecting thereto a power source; and an electrical interface connected to the arm distal end, wherein the arm distal end is configured to be connected with an electrically powered pollen attractor and the electrical interface is configured for electrical coupling between the power controller and the pollen attractor when connected to the arm distal end.

According to some embodiments of the present disclosure, at least the arm distal end has a first degree of freedom with respect to the support structure to allow a movement thereof at least in a first direction extending along the pollination path, wherein the first degree of freedom is at least partially constrained.

According to some embodiments of the present disclosure, movement of the arm distal end through the first degree of freedom is powered by a motor.

According to some embodiments of the present disclosure, the first degree of freedom allows the arm distal end to move in the first direction at least between a forward position and a backward position, the forward and backward positions defining at least partially the constraint of the first degree of freedom.

According to some embodiments of the present disclosure, the at least one mechanical arm is configured to be biased for the arm distal end to be at the forward position, wherein the arm distal end is configured to move into the backward position at least when the plant engaging component, when connected to the arm distal end, engages the plants during forward movement of the carrier along the pollination path.

According to some embodiments of the present disclosure, the at least one mechanical arm is connected to the support structure by a hinged connector configured to provide the first degree of freedom.

According to some embodiments of the present disclosure, the at least one mechanical arm is elastically resilient, thereby providing the first degree of freedom.

According to some embodiments of the present disclosure, at least the arm distal end has a second degree of freedom with respect to the support structure to allow a movement thereof at least in a second direction extending perpendicular to the pollination path as well as to the at least one mechanical arm, wherein the second degree of freedom is at least partially constrained.

According to some embodiments of the present disclosure, the second degree of freedom allows the arm distal end to move in the second direction at least between an upward position and a downward position, the upward and downward positions defining at least partially the constraint of the second degree of freedom.

According to some embodiments of the present disclosure, the at least one mechanical arm is connected to the support structure by a hinged connector configured to provide the second degree of freedom.

According to some embodiments of the present disclosure, the at least one mechanical arm is elastically resilient, thereby providing the second degree of freedom.

According to some embodiments of the present disclosure, the at least one mechanical arm at least partially defines a connection path for providing the electrical coupling between the arm distal end and the power controller.

According to some embodiments of the present disclosure, the pollination system comprises a pollen attractor electrically couplable to a power source and having a pollen-attracting surface configured to generate an electrostatic field by virtue of high voltage powered by the power source for attracting pollen, the pollen attractor being configured to be detachably attachable to the arm distal end.

According to some embodiments of the present disclosure, the pollen attractor comprises an electrode configured to generate the electrostatic field.

According to some embodiments of the present disclosure, the pollination cartridge is configured to be detachably attachable to the pollen attractor.

According to some embodiments of the present disclosure, the pollen adsorbing matrix is configured to collect the pollen attracted by the pollen attractor.

According to an aspect of some embodiments of the present disclosure, there is provided a pollination system comprising: a mounting position for a pollen adsorbing matrix; and an electrically powered pollen attractor comprising an insulated spiral conductor, configured to receive a high voltage and generate a pollen-attracting electrostatic field therefrom; wherein the electrostatic field transmits through the pollen adsorbing matrix when the pollen adsorbing matrix is mounted to the pollination system, and the pollen attractor receives the high voltage.

Unless otherwise defined, all technical and/or scientific terms used herein have the same meaning as commonly understood by one of ordinary skill in the art to which the present disclosure pertains. Although methods and materials similar or equivalent to those described herein can be used in the practice or testing of embodiments of the present disclosure, exemplary methods and/or materials are described below. In case of conflict, the patent specification, including definitions, controls. In addition, the materials, methods, and examples are illustrative only and are not intended to be necessarily limiting.

As will be appreciated by one skilled in the art, aspects of the present disclosure may be embodied as a system, method, or computer program product. Accordingly, aspects of the present disclosure may take the form of an entirely hardware embodiment, an entirely software embodiment (including firmware, resident software, microcode, etc.) or an embodiment combining software and hardware aspects that may all generally be referred to herein as a “circuit,” “module” or “system” (e.g., a method may be implemented using “computer circuitry”). Furthermore, some embodiments of the present disclosure may take the form of a computer program product embodied in one or more computer readable medium(s) having computer readable program code embodied thereon. Implementation of the method and/or system of some embodiments of the present disclosure can involve performing and/or completing selected tasks manually, automatically, or a combination thereof. Moreover, according to actual instrumentation and equipment of some embodiments of the method and/or system of the present disclosure, several selected tasks could be implemented by hardware, by software or by firmware and/or by a combination thereof, e.g., using an operating system.

For example, hardware for performing selected tasks according to some embodiments of the present disclosure could be implemented as a chip or a circuit. As software, selected tasks according to some embodiments of the present disclosure could be implemented as a plurality of software instructions being executed by a computer using any suitable operating system. In some embodiments of the present disclosure, one or more tasks performed in method and/or by system are performed by a data processor (also referred to herein as a “digital processor”, in reference to data processors which operate using groups of digital bits), such as a computing platform for executing a plurality of instructions. Instruction executing elements of the processor may comprise, for example, one or more microprocessor chips, ASICs, and/or FPGAs. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well. Any of these implementations are referred to herein more generally as instances of computer circuitry.

Any combination of one or more computer readable medium(s) may be utilized for some embodiments of the present disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device. A computer readable storage medium may also contain or store information for use by such a program, for example, data structured in the way it is recorded by the computer readable storage medium so that a computer program can access it as, for example, one or more tables, lists, arrays, data trees, and/or another data structure. Herein a computer readable storage medium which records data in a form retrievable as groups of digital bits is also referred to as a digital memory. It should be understood that a computer readable storage medium, in some embodiments, is optionally also used as a computer writable storage medium, in the case of a computer readable storage medium which is not read-only in nature, and/or in a read-only state.

Herein, a data processor is said to be “configured” to perform data processing actions insofar as it is coupled to a computer readable medium to receive instructions and/or data therefrom, process them, and/or store processing results in the same or another computer readable medium. The processing performed (optionally on the data) is specified by the instructions, with the effect that the processor operates according to the instructions. The act of processing may be referred to additionally or alternatively by one or more other terms; for example: comparing, estimating, determining, calculating, identifying, associating, storing, analyzing, selecting, and/or transforming. For example, in some embodiments, a digital processor receives instructions and data from a digital memory, processes the data according to the instructions, and/or stores processing results in the digital memory. In some embodiments, “providing” processing results comprises one or more of transmitting, storing and/or presenting processing results. Presenting optionally comprises showing on a display, indicating by sound, printing on a printout, or otherwise giving results in a form accessible to human sensory capabilities.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object-oriented programming language such as Java, Smalltalk, C++ or the like and conventional procedural programming languages, such as the “C” programming language or similar programming languages. Additionally or alternatively, sequences of logical operations (optionally logical operations corresponding to computer instructions) may be embedded in the design of an ASIC and/or in the configuration of an FPGA device. The program code may execute entirely on the user's computer, partly on the user's computer (e.g., as a stand-alone software package), partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the present disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer; and may not be feasible or practical for performing purely manually, by a human expert. A human expert who wanted to manually perform similar tasks, such inspecting objects, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the present disclosure are herein described, by way of example only, with reference to the accompanying drawings. With specific reference now to the drawings in detail, it is stressed that the particulars shown are by way of example, and for purposes of illustrative discussion of embodiments of the present disclosure. In this regard, the description taken with the drawings makes apparent to those skilled in the art how embodiments of the present disclosure may be practiced.

In the drawings:

FIG. 1 schematically illustrates a kit including a pollination cartridge, a pollinating device, and a pollen collector device, according to some examples of the presently disclosed subject matter;

FIGS. 2A-2C schematically illustrate the pollen collector device, according to some examples of the presently disclosed subject matter;

FIGS. 2D-2E schematically illustrate a variation of pollen collector device, according to some examples of the presently disclosed subject matter;

FIGS. 3A-3C schematically illustrate the pollination cartridge, according to some examples of the presently disclosed subject matter;

FIG. 4 schematically represents a flower, relating to some examples of the presently disclosed subject matter;

FIG. 5 schematically represents a distal portion of pollen collector device according to some examples of the presently disclosed subject matter;

FIG. 6A schematically represents a dehiscent (mature and split open) anther of a pistil, shedding pollen onto a pollen adsorbing matrix, according to some examples of the presently disclosed subject matter;

FIG. 6B schematically represents a stigma of a stamen, receiving pollen from a pollen adsorbing matrix, according to some examples of the presently disclosed subject matter;

FIGS. 7A-7B schematically illustrate respective methods for pollen collection and artificial pollination, according to some examples of the presently disclosed subject matter;

FIGS. 8A-8B illustrate a vehicle-drawn artificial pollination system, according to some examples of the presently disclosed subject matter;

FIGS. 9A-9C schematically illustrate a mechanical arm used in an artificial pollination system, according to some examples of the presently disclosed subject matter;

FIG. 9D is a block diagram schematically illustrating enclosed electronics of the mechanical arm, according to some examples of the presently disclosed subject matter;

FIG. 9E schematically illustrates the mechanical arm as a block diagram, according to some examples of the presently disclosed subject matter;

FIG. 10 schematically represents interaction of an artificial pollination system with a plant, according to some examples of the presently disclosed subject matter;

FIG. 11 is a block diagram schematically representing components, inputs, and other data structures of a controller for a powered mechanical arm, according to some examples of the presently disclosed subject matter;

FIGS. 12A-12B schematically represent movement of a pollen adsorbing matrix by a matrix transport of a powered mechanical arm, according to some examples of the presently disclosed subject matter;

FIG. 13 schematically represents a portion of an inflorescence protruding through a protective net into proximity with a portion of a pollen adsorbing matrix, according to some examples of the presently disclosed subject matter; and

FIG. 14 is a block diagram schematically representing components, inputs, and other data structures of a controller for a hand-held pollen collector device, according to some examples of the presently disclosed subject matter.

### DESCRIPTION OF SPECIFIC EMBODIMENTS OF THE DISCLOSURE

The presently disclosed subject matter, in some embodiments thereof, relates to the field of artificial pollination and more particularly, but not exclusively, to systems, devices, and methods for performing artificial pollination.

### Overview

A broad aspect of the presently disclosed subject matter relates to systems, device, components, and methods for artificial pollination. Operations of artificial pollination include one or more of: collecting pollen grains (interchangeably referred to herein as “pollen”) from male flower parts of plants (“source plants”), storing and/or transferring the collected pollen in/to another location and/or device, and releasing the pollen (“deploying” it, or “pollinating”) to female flower parts of plants (“target plants”). Solutions described herein relate to small- and/or large-scale artificial pollination for in-field/in-orchard applications. In particular, some examples comprise handheld devices for pollen collection and/or distribution; some examples comprise machine-drawn devices for pollen collection and/or distribution.

### Hand-Held Pollination Devices

According to a first aspect of the presently disclosed subject matter, there is provided a portable handheld pollination device configured to attract pollen, the device comprising: a power controller configured for electrically connecting thereto a power source; a pollen attractor electrically coupled to the power controller and having a pollen-attracting surface configured to generate an electrostatic field by virtue of high voltage powered by the power source for attracting the pollen; an insulation arrangement insulating at least the pollen attractor and the power controller from surroundings of the pollination device (e.g., its outdoor operating environment). In some examples, the pollen attractor can comprise an electrode configured to generate the electrostatic field.

In some examples, the insulation arrangement comprises a reach extender interconnecting the power controller and the pollen attractor. A proximal end of the device including the power controller is provided with a handle and at least one user-operated control (e.g., a button, slider, trigger, or other switch and/or setting device) to enable hand operation by an operator. The reach extender is sized to extend a height reach of an operator holding the handle for bringing the pollen attractor into approximation with a plant flower and/or inflorescence sufficient to attract pollen and/or brush pollen on to the plant.

The reach extender preferably is sized to allow the operator to reach flowers and inflorescences on a shrub or tree, beyond their normal reach.

The reach extender is, for example, at least 1 m long; optionally at least 1.5 m, 2 m, 3 m, 4 m, 5 m, 6 m, or longer. In some embodiments, the reach extender comprises an insulated rod or pole having an elongated hollow through which the power controller and power attractor are interconnected.

In some examples, the pollination device further comprises at least one floating grounding component electrically coupled, at least indirectly, to the power controller, and exposed for making grounding contacts with objects in the operating environment to enable grounding of excess charge accumulated on the portable handheld pollination device. The floating grounding component can have a conductive portion configured to be electrically exposed to the environment. In particular, plants may be used to provide an electrical ground connection.

According to a second aspect of the presently disclosed subject matter, there is provided a portable handheld pollination device configured to attract pollen, said device comprising: a power controller configured for electrically connecting thereto a power source; a pollen attractor electrically coupled to the power controller and having a pollen-attracting surface configured to generate an electrostatic field by virtue of high voltage powered by the power source for attracting the pollen; and at least one floating grounding component electrically coupled, at least indirectly, to the power controller to enable grounding of excess charge accumulated on the portable handheld pollination device, said floating grounding component having a conductive portion configured to be electrically exposed to surroundings of the pollination device.

In some examples, the pollination device can further comprise an insulation arrangement insulating at least the pollen attractor and the power controller from surroundings of the pollination device.

It is to be understood herein that the pollination device according to any of the first and second aspects can be a pollen exchanger, for example a pollen collecting device usable for collecting pollen from plants and a pollinating device, for example a pollination device usable for releasing pollen at plants.

It is to be understood herein that for the purposes of the present description, the pollen attractor is to be operated at a high voltage, which is to be understood to be above 1 kV, optionally up to 5 kV, 7 kV, 10 kV, 15 kV, or more; and a power source providing a voltage above 1 kV be considered as a high voltage power source. The high voltage can be provided to the pollen attractor by a high voltage power source such a battery providing a voltage above 1 kV, or by an electrical interface (such a voltage converter or transformed) connected to a power source providing a voltage lower than 1 kV and converting it into a high voltage of above 1 kV.

In some examples, the pollination device according to any of the first and second aspects of the presently disclosed subject matter can further comprise a proximal portion configured to be held by a user for operating the pollination device and a distal portion spaced apart from the proximal portion and comprising the pollen attractor, and the at least one floating grounding component can be positioned at the distal portion in proximity to the pollen attractor. The floating grounding component can be formed as an elastically resilient protrusion protruding from a location in proximity to the pollen attractor or as a frame. In some examples, the conductive portion can be configured to be electrically disposed to surroundings of the pollination device, and to engage the plant, at least when the pollen attractor attracts the pollen from the plant. The conductive portion can be configured to enable grounding of the excess charge accumulated on the portable handheld pollination device through the plant.

In some examples, the insulation arrangement of the pollination device according to any of the first and second aspects of the presently disclosed subject matter can comprise a first insulating material insulating at least partially the pollen attractor and a second insulating material insulating at least partially the power controller. The first insulating material can have a first dielectric strength and the second insulating material can have a second dielectric strength lesser than the first dielectric strength. In some examples, a coating of the first insulating material can have a first thickness and a coating of the second insulating material can have a second thickness greater than the first thickness. The first and/or second insulating material can be dielectric materials, and in some examples can be selected from a group comprising PTFE tube, Delrin, polycarbonate, fiberglass, Kapton, Formex, and combinations thereof.

In some examples, the pollination device according to any of the first and second aspects of the presently disclosed subject matter can comprise one or more of a handle configured to be held by a user for operating the device, an reach extender extending between the handle and the pollen attractor and having a length ranging between 1 m to 6 m, one or more LEDs indicating an operative state of the device, and one or more solder cables electrically connecting the power controller and the pollen attractor. In some examples, each of the handle, reach extender, LEDs, and solder cables can be insulated by the insulation arrangement.

According to a third aspect of the presently disclosed subject matter, there is provided a pollination cartridge for use with a pollination device, the pollination cartridge being configured to be detachably attachable to the pollination device and comprising a pollen adsorbing matrix configured to collect pollen from a source plant, the pollen adsorbing matrix being configured to independently store the collected pollen at least after the pollination cartridge is detached from the pollination device. In some examples, the pollination cartridge according to the third aspect of the presently disclosed subject matter can be configured to be used with the pollination device according to the first aspect of the presently disclosed subject matter.

In some examples, the pollen adsorbing matrix can comprise a dielectric material including an electrical insulating material; for example, PVC, nylon, polyethylene, polyester, PTFE, or another polymer. The pollen adsorbing matrix is configured to release the collected pollen to parts of flowers receptive to pollination upon suitable operation, for example, by wiping off the pollen therefrom. In some examples, the pollen adsorbing matrix can be elastically resilient for flexing upon engaging the plants.

The pollination cartridge can comprise one or more attachment elements configured for detachably attaching the pollination cartridge to the pollination device. The pollination device can be a pollen collector device for collecting the pollen and a pollinating device for releasing the pollen to perform pollination. A majority of the one or more attachment elements can be disposed at a periphery of the pollination cartridge.

In some examples, a thickness of the pollen adsorbing matrix can range between 0.1 mm to 50 mm. In some examples, the thickness is in a range suitable for a flexible solid sheet or woven sheet (textile), e.g., between about 0.1 mm to about 5 mm, or about 0.5 mm to about 2 mm. In some examples, a dielectric strength of the dielectric material can range between 1 kV/mm to 1000 kV/mm. In some examples, a ratio between the dielectric strength and the thickness can range between 0.3 kV/mm² to 800 kV/mm².

In some examples, the pollination device according to the first aspect of the presently disclosed subject matter can include the pollination cartridge according to any example of the third aspect of the presently disclosed subject matter.

According to a fourth aspect of the presently disclosed subject matter, there is provided a kit for artificial pollination, the kit comprising: at least one pollination device configured for assisting in at least one of collecting and pollinating; at least one pollination cartridge configured to be detachably attachable to the at least one pollination device, the at least one pollination cartridge comprising a pollen adsorbing matrix configured to at least one of collect the pollen, independently store the collected pollen, and pollinate (provide the collected and/or stored pollen to plants).

In some examples, the at least one pollination cartridge can comprise the pollination cartridge according to any example of the third aspect of the presently disclosed subject matter.

In some examples, the at least one pollination device can comprise the pollination device according to any example of the first aspect of the presently disclosed subject matter.

In some examples, the at least one pollination device can comprise a pollinating device configured for the at least one pollination cartridge to be detachably attached thereto for pollinating using the pollen. In some examples, the at least one pollination device can comprise at least two pollination devices, at least one of the at least two pollination devices being a pollen collector device and at least one of the at least two pollination devices being a pollinating device. The pollination cartridge can be configured to be detachably attachable to each of the at least two pollination devices. For instance, the pollination cartridge can be configured to be detachably attachable to the at least one pollen collector device for collecting the pollen, and to the at least one pollinating device used to pollinate plants with the pollen.

### Towed Pollination Devices

According to a fifth aspect of the presently disclosed subject matter, there is provided a pollination system mountable on a carrier configured to carry the system in a field along a pollination path alongside plants to enable the system to assist in pollination of the plants, the system comprising: a support structure; a power controller configured for electrically connecting thereto a power source; and at least one mechanical arm having an arm proximal end connected to the support structure and an arm distal end configured to be connected with an electrically powered pollen attractor; and an electrical interface connected to the arm distal end and configured for electrically coupling the power controller and the pollen attractor when connected to the arm distal end; wherein at least the arm distal end has a first degree of freedom with respect to the support structure to allow a movement thereof at least in a first direction extending along the pollination path, wherein the first degree of freedom is at least partially constrained. In some examples, the at least one mechanical arm can at least partially define a connection path for providing the electrical coupling between the arm distal end and the power controller.

In some examples, the pollination system according to the fifth aspect of the presently disclosed subject matter can comprise an electrically powered pollen attractor connected at the arm distal end and in electrical connection with the at least one interface. The pollen attractor can have a pollen-attracting surface configured to generate an electrostatic field by virtue of high voltage provided by the power source for attracting the pollen. The pollen attractor can comprise an electrode configured to generate the electrostatic field.

According to a sixth aspect of the presently disclosed subject matter, there is provided a pollination system mountable on a carrier configured to carry the system in a field along a pollination path alongside plants to enable the system to assist in pollination of the plants, the system comprising: a support structure; at least one mechanical arm having an arm proximal end connected to the support structure and an opposite arm distal end; and a pollen attractor electrically couplable to a power source and having a pollen-attracting surface configured to generate an electrostatic field by virtue of high voltage provided by the power source for attracting pollen, the pollen attractor being configured to be detachably attachable to the arm distal end; wherein at least the arm distal end has a first degree of freedom with respect to the support structure to allow a movement thereof at least in a first direction extending along the pollination path, wherein the first degree of freedom is at least partially constrained.

According to a seventh aspect of the presently disclosed subject matter, there is provided a pollination system mountable on a carrier configured to carry the system in a field along a pollination path alongside plants to enable the system to assist in pollination of the plants, the system comprising: a support structure; and a mechanical arm having an arm proximal end connected to the support structure and an arm distal end; and a pollination cartridge connected, at least indirectly, to the arm distal end, the pollination cartridge comprising a pollen adsorbing matrix configured to engage the plants, the pollen adsorbing matrix being elastically resilient to flex in a direction extending at least partially along the pollination path at least upon engaging the plant.

In some examples, the pollination system according to the seventh aspect of the presently disclosed subject matter can comprise a pollen attractor electrically couplable to a power source and having a pollen-attracting surface configured to generate an electrostatic field by virtue of high voltage provided by the power source for attracting pollen. The pollen attractor can be configured to be detachably attachable to the arm distal end and/or can comprise an electrode configured to generate the electrostatic field.

It is to be understood herein that the pollen attractor of the pollination system according to any of the fifth, sixth, and seventh aspects of the presently disclosed subject matter can correspond to, and can include some or all of the features of, the pollen attractor of the pollination device according to any example of the first aspect of the presently disclosed subject matter.

In some examples, the pollination system according to any of the fifth and sixth aspects of the presently disclosed subject matter can further comprise a pollination cartridge connected, at least indirectly, to the arm distal end, and the pollination cartridge can comprise a pollen adsorbing matrix configured to engage the plants. In some examples, the pollination cartridge of the pollination system according to any of the fifth, sixth, and seventh aspects of the presently disclosed subject matter can be connected to the pollen attractor, which is connected to the arm distal end. In some examples, the pollination cartridge can correspond to, and can include some or all of the features of, the pollination cartridge according to any example of the third aspect of the presently disclosed subject matter.

In some examples, the pollen adsorbing matrix (of the pollination cartridge) of the pollination system according to any of the fifth, sixth, and seventh aspects of the presently disclosed subject matter can be elastically resilient to flex in a direction extending at least partially along the pollination path at least upon engaging the plant. The pollination cartridge can be configured to be detachably attachable, at least indirectly, to the arm distal end. In some examples, at least a portion of the pollen adsorbing matrix can be reversibly bendable in a direction extending along the pollination path and/or the pollination can be formed to be smooth to prevent the plants from damage when the pollen adsorbing matrix engages and/or contacts the plants.

In some examples, the pollen adsorbing matrix (of the pollination cartridge) of the pollination system according to any of the fifth, sixth, and seventh aspects of the presently disclosed subject matter can correspond to, and can include some or all of the features of, the pollen adsorbing matrix of the pollination cartridge according to any example of the third aspect of the presently disclosed subject matter.

In some examples, the pollination system according to the seventh aspect of the presently disclosed subject matter can further comprise: a power controller configured for electrically connecting thereto a power source; and an electrical interface connected to the arm distal end, wherein the arm distal end is configured to be connected with an electrically powered pollen attractor and the electrical interface is configured for electrically coupling the power controller and the pollen attractor when connected to the arm distal end. The at least one mechanical arm can at least partially defines a connection path for providing the electrical coupling between the arm distal end and the power controller.

In some examples, the at least one mechanical arm of the pollination system according to the seventh aspect of the presently disclosed subject matter can have a first degree of freedom with respect to the support structure to allow a movement thereof at least in a first direction extending along the pollination path. The first degree of freedom can be at least partially constrained.

In some examples, the at least one mechanical arm of the pollination system according to any of the fifth, sixth, and seventh aspects of the presently disclosed subject matter, the first degree of freedom allows the arm distal end to move in the first direction at least between a forward position and a backward position. The forward and backward positions can define at least partially the constraint of the first degree of freedom. The at least one mechanical arm can be configured to be biased for the arm distal end to be at the forward position, wherein the arm distal end is configured to move into the backward position at least when the arm distal end, at least indirectly (for example via the pollen attractor and/or the pollination cartridge connected thereto), engages the plants during forward movement of the carrier along the pollination path. In some examples, the at least one mechanical arm can be connected to the support structure by a connection element configured to provide the first degree of freedom. In some examples, the at least one mechanical arm can be elastically resilient, thereby providing the first degree of freedom.

In some examples, the at least one mechanical arm of the pollination system according to any of the fifth, sixth, and seventh aspects of the presently disclosed subject matter, can have has a second degree of freedom with respect to the support structure to allow a movement thereof at least in a second direction extending perpendicular to the pollination path as well as to the at least one mechanical arm. The second degree of freedom can be at least partially constrained. The second degree of freedom allows the arm distal end to move in the second direction at least between an upward position and a downward position. The upward and downward positions can define at least partially the constraint of the second degree of freedom. In some examples, the at least one mechanical arm can be connected to the support structure by a connection element configured to provide the second degree of freedom. In some examples, the at least one mechanical arm can be elastically resilient, thereby providing the second degree of freedom.

### Further Aspects of Towed and Hand-Held Pollination Devices

According to an eighth aspect of the presently disclosed subject matter, there is provided a pollination system mountable on a carrier configured to carry the system along a pollination path alongside plants to enable the system to assist in pollination of the plants, the system comprising: a support structure; at least one mechanical arm, attached to the support structure; and a pollen exchanger, attached to the arm at a position which engages in contacts with the plants as the system moves along the pollination path; wherein the pollen exchanger comprises a matrix transport which receives a pollen adsorption matrix; and wherein the matrix transport operates to transport portions of the pollen adsorption matrix across the pollen exchanger to refresh a plant-engaging surface defined on a side of the pollen exchanger facing forward along the pollination path.

According to a ninth aspect of the presently disclosed subject matter, there is provided a pollination system mountable on a carrier configured to carry the system along a pollination path alongside plants to enable the system to assist in pollination of the plants, the system comprising: a support structure; at least one mechanical arm, attached to the support structure, and positioning a plant-engaging surface of a pollen exchanger where the plant-engaging surface engages in contacts with the plants as the system moves along the pollination path; a motor, attached to the at least one mechanical arm, and operable to change at least an angle at which the plant-engaging surface contacts the plants; and a controller, configured to operate the motor to adjust the angle in response to one or more indications of force exerted upon the plants by the contacts.

According to a tenth aspect of the presently disclosed subject matter, there is provided a portable handheld pollination device configured to attract pollen, said device comprising: a power controller at a handheld proximal end of the pollination device, operated through at least one user-operated control, and configured to electrically connect a power source thereto; a pollen attractor electrically coupled to the power controller via an interconnecting reach extender, and having a pollen-attracting surface, wherein the pollen attractor is configured to generate an electrostatic field by virtue of high voltage powered by the power source for attracting the pollen; a user interface; and a processor and memory, wherein the memory stores instructions which instruct the processor to: access one or more indications of operation of the pollination device, evaluate the indications according to one or more criteria of targeted device operation, and operate the user interface to provide one or more indications providing guidance regarding operation of the pollination device, based on the evaluation.

According to an eleventh aspect of the presently disclosed subject matter, there is provided a pollination system comprising: a pollen exchanger carrying a pollen adsorption matrix; wherein the pollen exchanger includes a size-selective barrier extending across the pollen adsorption matrix; and the size-selective barrier has apertures sized to restrict which sizes of plant parts are able to contact the pollen adsorption matrix.

According to a twelfth aspect of the presently disclosed subject matter, there is provided a portable handheld pollination device configured to attract pollen, said device comprising: a handle, sized to be held by an operator; a pollen exchanger; and a reach extender interconnecting the handle and the pollen exchanger; wherein the pollen exchanger comprises a matrix transport which receives a pollen adsorption matrix; and wherein the matrix transport operates to transport portions of the pollen adsorption matrix across the pollen exchanger to refresh a plant-engaging surface defined on a side of the pollen exchanger which exposes the pollen adsorption matrix to plant contacts.

According to a thirteenth aspect of the presently disclosed subject matter, there is provided a fabric used as a pollen adsorbing matrix for pollen collection, the fabric comprising a woven polymer of about 300 Denier or coarser, wherein the fabric is mounted on a pollination device with a first side of the fabric facing a pollen attractor configured to generate an electrostatic field of at least 1 kV, and a second side presenting a pollen collecting surface to collect pollen.

Before explaining at least one embodiment of the present disclosure in detail, it is to be understood that the present disclosure is not necessarily limited in its application to the details of construction and the arrangement of the components and/or methods set forth in the following description and/or illustrated in the drawings and/or given in the Examples. drawings. Features described in the current disclosure, including features of the presently disclosed subject matter, are capable of other embodiments or of being practiced or carried out in various ways.

### Hand-Held Pollination Devices

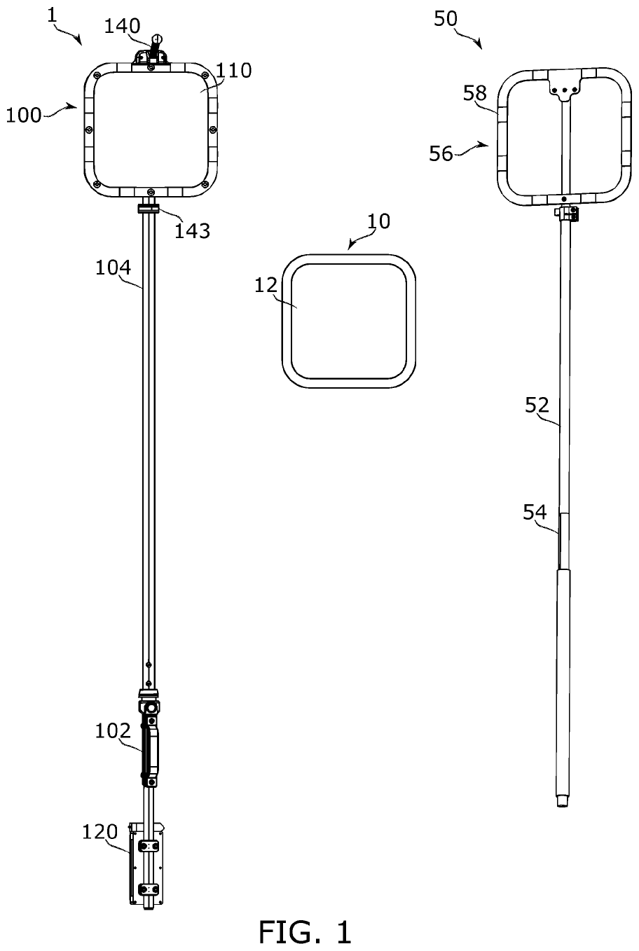

Reference is now made to FIG. 1, which schematically illustrates a kit 1 including a pollination cartridge 10, a pollinating device 50, and a pollen collector device 100, according to some examples of the presently disclosed subject matter. The pollinating device 50 and the pollen collector device 100 constitute portable and handheld pollination devices and the pollination cartridge is configured to be detachably attachable, for example by attachment means described in detail herein below, selectively to the pollinating device 50 and the pollen collector device 100. It is to be understood herein that although both the pollination devices, i.e., pollen collector device 100 and the pollinating device 50 have been illustrated herein as being included in the kit 1, in some examples, the kit 1 can comprise either of the pollination devices along with the pollination cartridges 10. Also, although only one each of the pollen collector device 100, the pollination cartridge 10, and the pollinating device 50 have been illustrated herein, the kit 1 can include any number of the pollen collector devices 100, any number of the pollination cartridges 10, and any number of the pollinating devices 50. The number of the pollination devices and the pollination cartridges can be same or different. For example, the kit 1 can include one (or any other number) pollen collector device 100, fifty (or any other number) pollination cartridges 10, and five (or any other number) pollinating devices 50.

The pollination cartridge 10 includes a pollen adsorbing matrix 12 configured for pollen to stick thereto and subsequently be wiped therefrom as appropriate; e.g., wiped to a storage container, and/or wiped from pollen adsorbing matrix 12 directly to the stamen of a flower (that is, parts of a flower receptive to pollination). For instance, the pollination cartridge 10 can be attached to the pollen collector device 100 and can be used to collect pollen onto the pollen adsorbing matrix 12 (for example, as described in detail herein further below), and then the pollination cartridge 10 can be detached from the pollen collector device 100 while still holding the pollen on the pollen adsorbing matrix 12 and can be used to store and/or transfer the pollen to a desired location. Subsequently, the pollination cartridge 10 (storing the pollen on the pollen adsorbing matrix 12) can be attached to the pollinating device 50 and can be used to release pollen to parts of flowers receptive to pollination, for example at female parts of the plants. In some examples, the pollen can be wiped off the pollen adsorbing matrix 12 for releasing pollen as appropriate, e.g., transferring pollen from pollen adsorbing matrix 12 directly to a flower.

The pollinating device 50 has an elongated structure to enable the user to release the pollen at the desired location at the plant. For instance, the pollinating device 50 comprises an extension element 52 having a handle 54 to be held by the user at its proximal end and a pollination cartridge support 56 for attaching thereto the pollination cartridge 10 at its distal end. The pollination cartridge support 56 has connectors 58 configured to detachably attach to corresponding connectors of the pollination cartridge 10. In the illustrated example, the connectors 58 have been illustrated to be adhesive elements. In other examples, the connectors 58 can be any other suitable connectors to enable easy and quick attachment and detachment of the pollination cartridge to the pollinating device 50. It is to be understood herein that the pollination cartridge support 56 has been illustrated as having a frame-like shape similar to that of the pollination cartridge 10, however, the pollination cartridge support can have any other suitable shape and size for the pollination cartridge to be detachably attached thereto and release the pollen (use the pollen to pollinate a plant).

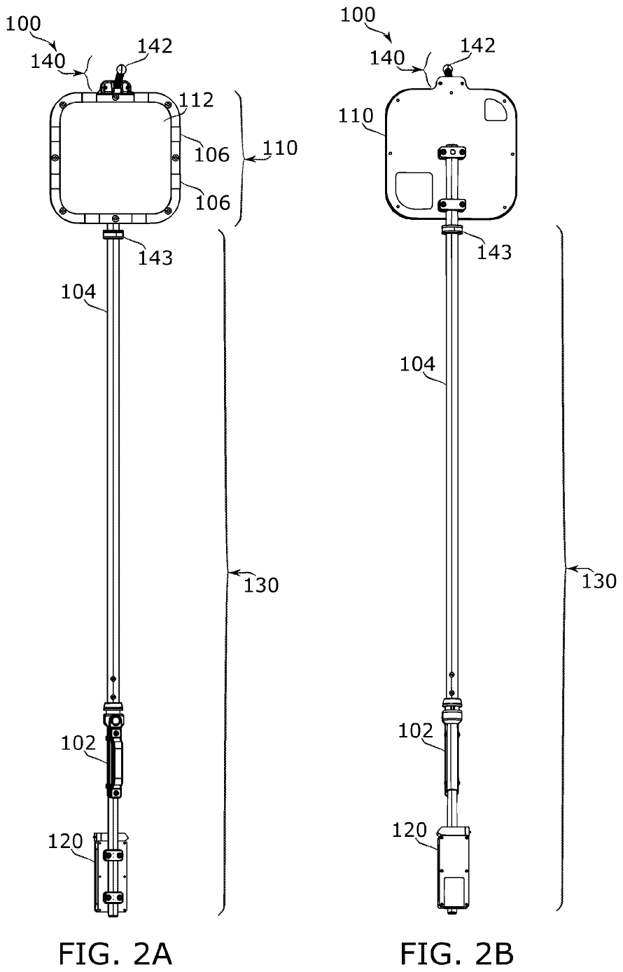

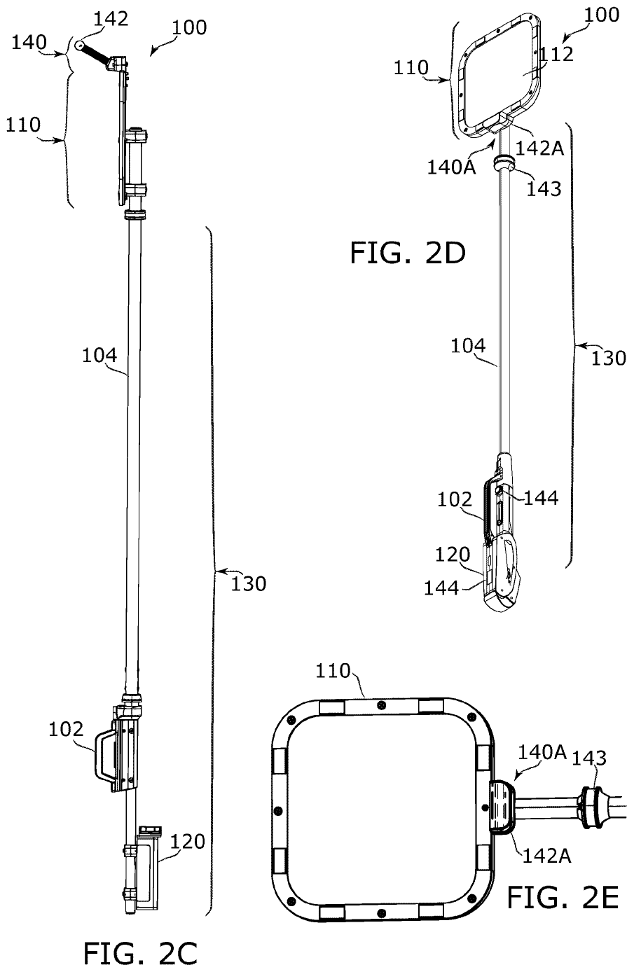

The pollen collector device 100 and the pollination cartridge 10 have been described herein with reference to FIGS. 2A to 2C and FIGS. 3A-3C respectively. It is to be understood herein that FIGS. 2A-2C and 3A-3C illustrate and the corresponding description below detail several features of the pollen collector device 100 and the pollination cartridge 10, however, in some examples of the presently disclosed subject matter, the pollen collector device 100 and/or the pollination cartridge 10 can be realized without some of the features thereof described herein below. It is to be further understood herein that although the device 100 has been described herein as being a pollen collector device, it can be used as pollinating device or a pollen holding device for holding the pollen as well, and accordingly be also considered as a pollinating device 50. In other words, it is to be understood herein that the terms the pollen collector device and the pollinating device can be interchangeably used herein to refer to the device 100.

### Pollen Collecting and Pollination

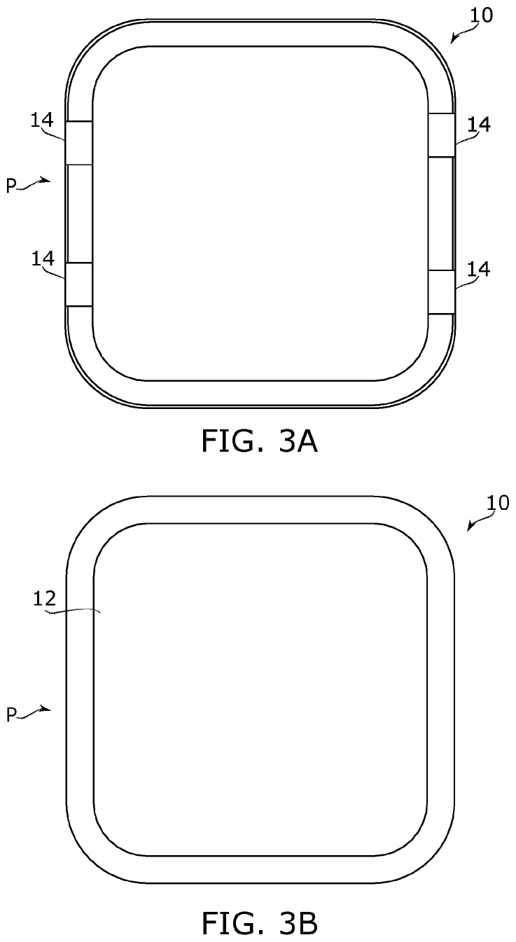

Brief reference is now made to FIG. 4, which schematically represents a flower 400, relating to some examples of the presently disclosed subject matter. Flower 400 may be the flower of any flowering plant. The example shown depicts a flower of an avocado plant. Petals 406 are shown open. Pistils 408 are male (pollen-providing) structures of flower 400. Stamen 410 is a female (pollen receiving) structure of flower 400.

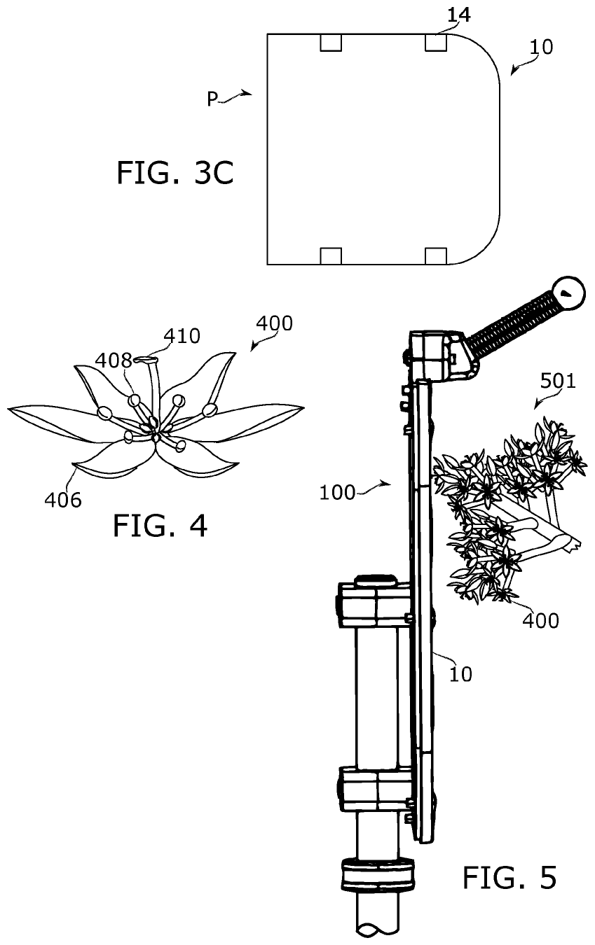

Further reference is made to FIG. 5, which schematically represents a distal portion of pollen collector device 100 according to some examples of the presently disclosed subject matter. As depicted, pollination cartridge 10 has been brought into contact with inflorescence 501.

Inflorescence 501 comprises a plurality of flowers 400, each in its own particular state of maturity. In some plants (e.g., avocado plant cultivars), flowers 400 progress through one or both of a “functionally male” (pollen-producing) state and a “functionally female” (pollen-receptive) state. These states potentially exist simultaneously within a single flower, within a single plant, and/or between the same or different cultivars. However, this is not necessarily the case; or if it is the case, the overlap may be for a period so brief that it limits the pollination effectiveness. In avocados, for example, functionally male and functionally female periods typically occur each day, but flowers of a single cultivar may succeed to being functionally female only after and/or near the end of their functionally male period, limiting the efficiency of self-fertilization. While this is potentially addressed by co-cultivation of cultivars with different flower maturation times, this constraint potentially limits cultivar choice for growers—and it may still happen that time-varying cultivation conditions such as temperature, wind directions, and/or plant stress disrupt synchronization.

Artificial fertilization potentially provides an option which allows greater offsets in time and/or space between pollen production and pollination itself, and/or increases pollination efficacy.

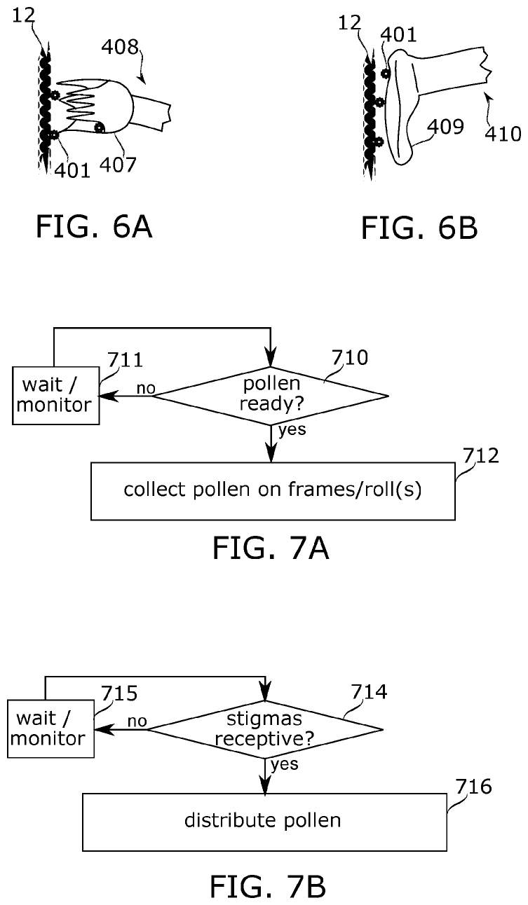

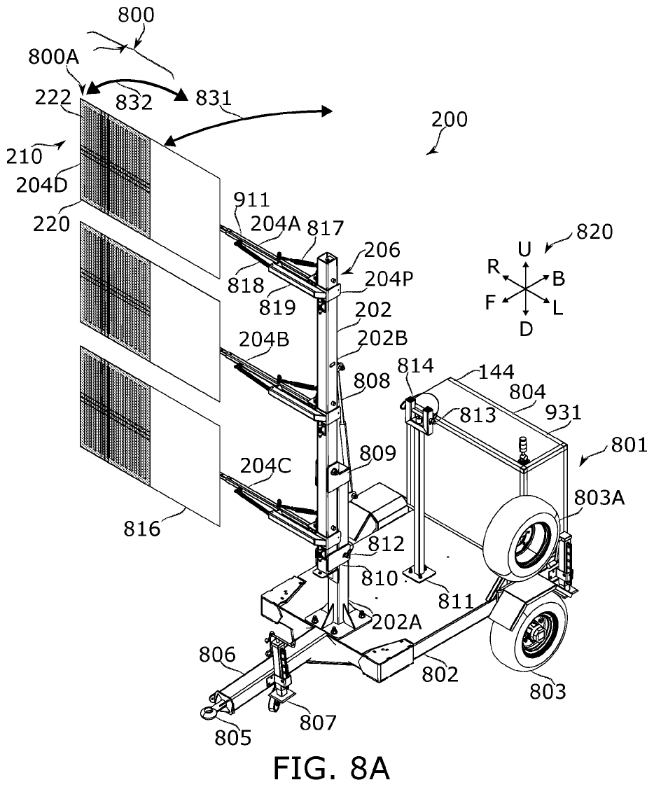

Reference is now made to FIG. 6A, which schematically represents a dehiscent (mature and split open) anther 407 of a pistil 408, shedding pollen 401 onto a pollen adsorbing matrix 12, according to some examples of the presently disclosed subject matter. Reference is also made to FIG. 6B, which schematically represents a stigma 409 of a stamen 410, receiving pollen 401 from a pollen adsorbing matrix 12, according to some examples of the presently disclosed subject matter. Further reference is made to FIGS. 7A-7B, which schematically illustrate respective methods for pollen collection and artificial pollination, according to some examples of the presently disclosed subject matter.

At block 710 (FIG. 7A), in some examples, a determination is made according to a condition used to estimate the readiness of pollen produced by plants from which pollen is to be collected. For example, this determination may be made according to a time of day at which the flowers of a certain cultivar are known and/or estimated to have brought their pollen to sufficient readiness to be viably used in pollination after collection. Optionally pollen is considered ready for another reason, e.g., for study, consumption, or use in a product.

Optionally, measurements and/or observations are used in determination of readiness; e.g., measurements of environmental factors such as temperature and light exposure. Optionally, the measurements include direct observation of plant flower state, e.g., imaging of plant flower opening times and/or other morphological changes in flowers over time, such as indications of anther dehiscence. Optionally, some form of pollen sampling is used, such as airborne pollen sampling. If the pollen is mature or otherwise considered suitably available, the flowchart continues with block 712. Otherwise, the flowchart enters a state of further waiting and/or monitoring at block 711, looping back to block 710 until pollen is deemed to be available for collecting. It should be understood that the method of FIG. 7A optionally begins with waiting and/or monitoring operations of block 711 rather than proceeding immediately to the determination of block 710.

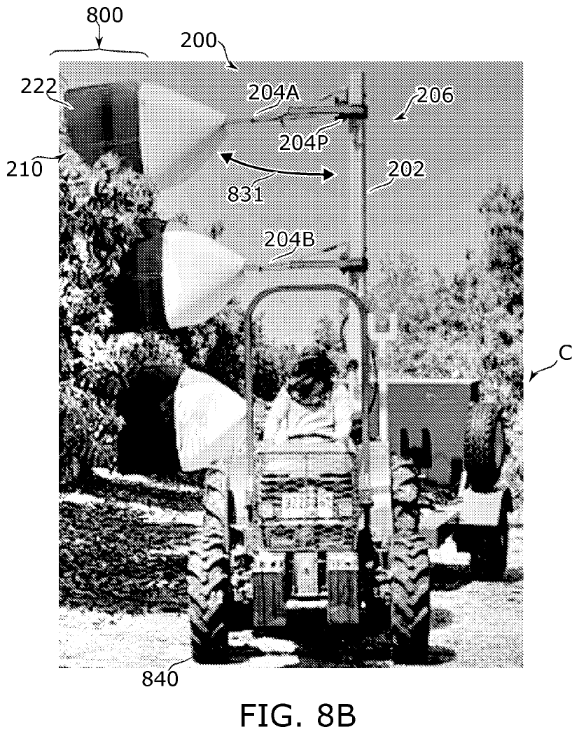

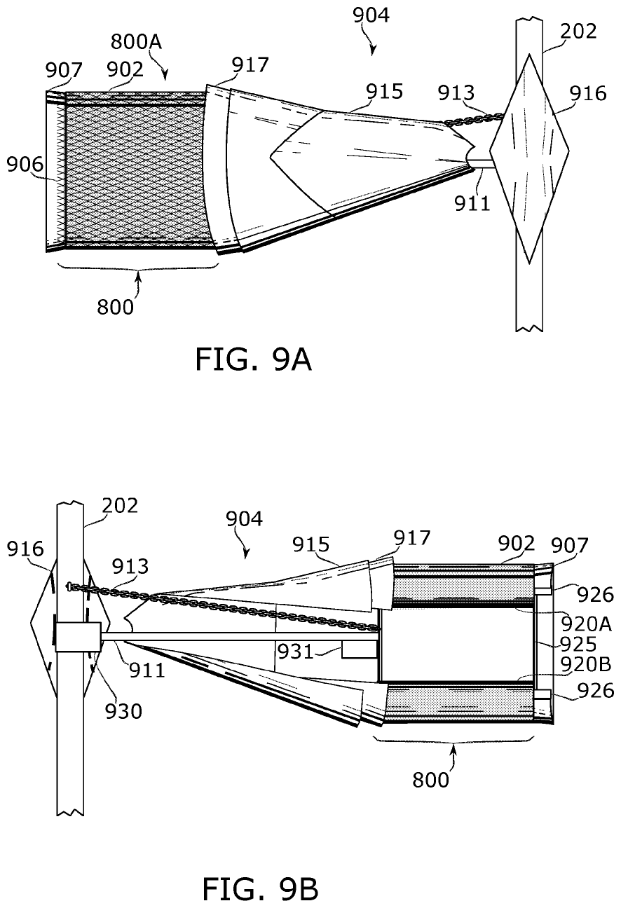

At block 712, in some examples, pollen is collected. FIG. 6A represents an example of this operation in which pollen is collected from a flower onto a pollen adsorbing matrix 12. Pollen adsorbing matrix 12 comprises a dielectric material in sheet form, optionally implemented as a textile positioned in front of an electrostatically charged pollen attractor 110. For example, it may be implemented as described in relation to pollination cartridge 10 (e.g., of FIGS. 1 and 3A-3C), pollination cartridge 220 (e.g., of FIGS. 8A-8B), and/or adsorbing matrix 902 (e.g., of FIGS. 9A-9E). Upon reaching a sufficient proximity to a source of pollen 401, for example, a flower 400 (e.g., proximity to anther 407), electrostatic forces developed by pollen attractor 110 attract grains of pollen 401 to pollen adsorbing matrix 12. Optionally, mechanical disturbance of anther 407 by contact with pollen adsorbing matrix 12 encourages anther 407 to dehisce (open), releasing pollen 401 to be adsorbed.

Once adsorbed, pollen 401 remains loosely attached to pollen adsorbing matrix 12 until disturbed, e.g., mechanically disturbed.

At block 714 (FIG. 7B), in some examples, it is determined whether or not flowers to be pollinated are in a “functionally female” state, e.g., a state in which stigmas of the flowers are developed, exposed to receive pollen, and still healthy. The determination may be made on the basis of known and/or estimated state, substantially as described in relation to block 710 and pollen availability for collection, adjusted as appropriate for the assessment of flower receptiveness to pollination. If the stigmas are receptive, the flowchart proceeds with the distribution of pollen (pollination) at block 716. Until the condition of readiness to be pollinated is met, the flowchart enters a state of waiting and/or monitoring at block 715, optionally cycling between blocks 714, 715 until the functionally female state is determined to exist. It should be understood that the method of FIG. 7B optionally proceeds to the waiting/monitoring operations of block 715 before the determination of block 714.

FIG. 6B represents an operation of artificially pollinating a flower, e.g., as an example of the operations of block 716. For effective pollination, it is a potential advantage for the pollen 401 to be adsorbed superficially upon pollen adsorbing matrix 12, e.g., since in this position it is well-exposed to being mechanically dislodged upon contact with stigma 409, or another flower part.

### Components of Hand-Held Pollination Devices

Reference is now made to FIGS. 2A-2C, which schematically illustrate the pollen collector device 100, according to some examples of the presently disclosed subject matter. Reference is also made to FIGS. 2D-2E, which schematically illustrate a variation of pollen collector device 100, according to some examples of the presently disclosed subject matter. The pollen collector device 100 is a portable and handheld device to be held by a user for its operation. The user can carry the pollen collector device 100 in hand while operating across a field/orchard of plants collecting pollen therefrom. The pollen collector device 100 comprises a pollen attractor 110 configured to attract pollen, for example, from the plants. The pollen attractor 110 comprises an electrode configured to generate electrostatic field by virtue of a high voltage provided thereto. In general, the pollen attractor 110 can be electrically coupled to a high voltage power source and generates an electrostatic field by virtue of the high voltage provided by the high voltage power source. The electrostatic field causes the pollen to be attracted towards the pollen attractor 110 when the pollen attractor is positioned in proximity to the plants. The pollen collector device 100 further includes a power controller 120 configured for electrically connecting to a power source and to conduct voltage therefrom to the pollen attractor 110. It is to be understood herein that for the purposes of the present description, the pollen attractor is to be operated at a high voltage, which is to be understood to be above 1 kV, and a power source providing a voltage above 1 kV be considered as a high voltage power source. The high voltage (above 1 kV) can be provided to the pollen attractor by a high voltage power source such a battery providing a voltage above 1 kV, or by an electrical interface (such a voltage converter or transformed) connected to a power source providing a voltage lower than 1 kV and converting it into a high voltage of above 1 kV. In some examples, voltages are higher, e.g., specified to reach at least 5 kV, at least 7 kV, at least 10 kV, at least 15 kV, or another voltage. Actual potential differences with the environment may be smaller, particularly for floating-ground embodiments, although periodic connection to an environmental ground (e.g., by touching a plant or other pathway to the earth with a grounding electrode) may restore higher potential differences. In some examples, the power source can be internal such as a battery. In some examples, the power source can be external such as provided via an electrical interface. It is to be understood herein that the power controller 120 can be constituted by any component or unit configured to enable electrical coupling between the power source and the pollen attractor 110. For instance, in some examples, the power controller 120 can constitute a part of an electrical interface between a power source (whether internal or external; for example a battery) and the pollen attractor 110. In some examples, the power controller 120 can constitute a part of the pollen attractor 110. Herein, the distal end including pollen attractor 110 is optionally referred to as a pollen exchanger, with or without an installed pollination cartridge 10.

The pollen collector device 100 further includes an insulation arrangement 130 insulating the pollen attractor 110 and the power controller 120. The insulation arrangement 130 prevents a discharge of voltage (electrical potential) from the pollen attractor 110 when it approaches close to the plants and to prevent the user from an electric shock. For instance, the insulation arrangement 130 prevents (or at least significantly reduces) the corona effect between the pollen collector device 100 and the plants and/or the user.

In general, the insulation arrangement 130 can include a first insulating material having a first dielectric strength insulating the pollen attractor 110. In some examples, the first insulating material can include a dielectric material, and in particular example, can be selected from a group comprising PTFE tube, Delrin, polycarbonate, fiberglass, Kapton, Formex, and combinations thereof. The insulation arrangement 130 can include a second insulating material having a second dielectric strength insulating the power controller 120. In some examples, the second insulating material can include a dielectric material, and in particular example, can be selected from a group comprising PTFE tube, Delrin, polycarbonate, fiberglass, Kapton, Formex, and combinations thereof. In some examples, the dielectric strength of the first insulating material can be greater than the dielectric strength of the second insulating material.

The first insulating material can have a coating of thickness ranging between 1 micron to 5 cm on the pollen attractor 110. In some examples, the thickness of the coating of the first insulating material can be variable along the pollen attractor 110. The second insulating material can have a coating of thickness ranging between 1 micron to 5 cm on the power controller 120. It is to be understood herein that a combination of the dielectric strength and thickness of the first and/or the second insulating materials is chosen to prevent the leakage of electric current from the pollen attractor 110 and the power controller 120 while allowing the electrostatic field generated by the pollen attractor 110 to pass through to attract the pollen. For instance, an insulation property of the insulation arrangement depends on the combination of the dielectric strength of the insulating material and thickness of the coating used. This insulation property of the insulation arrangement 130 is selected to prevent the leakage of electric current from the pollen attractor 110 and the power controller 120 while allowing the electrostatic field generated by the pollen attractor 110 to pass through to attract the pollen. Accordingly, a ratio of the dielectric strength of the insulation material and thickness of its coating is selected to achieve the most effective insulation while maintaining the pollen-attracting capabilities of the pollen collector device 100.

In general, the pollen collector device 100 can include further components such as extension element to increase the reach of the user to taller/faraway plants, handle for being held by the user, switches for the user to control the operation of the device, and/or solder cables (cables with low resistance connection such as soldered connections) to conduct electricity from a power source or power controller to the pollen attractor. Some or all of these components can be insulated by the insulation arrangement by same or different insulating materials and/or by same or different insulating properties.

In the illustrated example, the pollen collector device 100 comprises the handle 102 configured for being held by the user for operating the pollen collector device 100. The handle 102 is positioned at a proximal (towards the user) portion of the pollen collector device 100 and the pollen attractor 110 is positioned at a distal (to be held towards the plants and away from the user) portion of the pollen collector device 100. The user can hold the handle 102 and position the pollen attractor 110 in proximity to the plants to operate the pollen collector device 100. The reach extender 104 extends between the proximal portion (for example, the handle 102) and distal portion (for example, the pollen attractor 110) of the pollen collector device 100 and defines the reach of the user to collect the pollen. The length of the reach extender can range between 1 m to 6 m according to the requirement of the user and/or according to the height of the plants. The pollen collector device 100 can further include LED(s) 143 or other indicators for indicating various operative states of the pollen collector device 100.

In the illustrated example, the pollen collector device 100 comprises solder cables (not shown) electrically connecting the power controller 120 to the pollen attractor 110. The insulation arrangement 130 insulates each of these components including the handle 102, the reach extender 104, the LEDs (not shown), and the solder cables (not shown). The insulation arrangement 130 insulates all these components thereby preventing the user from electric shock and leakage of electric current.

The insulation arrangement can comprise same or different insulating materials to insulate the above-described components of the pollen collector device 100. According to a specific example, Table 1 lists some of the insulating materials that can be used to insulate the components of the pollen collector device 100 along with their respective dielectric strengths and thickness of the coatings used.

TABLE 1
Insulating material breakdown voltage examples

| | | | | CALCULATED | |
|---|---|---|---|---|---|
| | | DIELECTRIC | | FOR GIVEN | |
| MATERIAL | DIMENSIONS | STRENGTH | | DIMENSIONS | |
| PTFE Tube | 6(ID)X8(OD) - | 80 | kV/mm | 80 | kV |
| | 1 mm thickness | | | | |
| Delrin | at least 2 mm | 20 | kV/mm | 40 | kV |
| Polycarbonate | 3 mm | 252 | kV/mm | 756 | kV |
| Fiberglass | 28 × 32; | 35 | kV/inch~ | 2.75 | kV |
| | 2 mm thickness | 1.37 | kV/mm | | |

In some examples, the insulating material for insulating all or some of these components can include a dielectric material, and in particular example, can be selected from a group comprising PTFE tube, Delrin, polycarbonate, fiberglass, Kapton, Formex, and combinations thereof.

In the illustrated example, the power controller 120 has been shown to be positioned at the distal portion of the pollen collector device 100, whereas in some examples, the power controller 120 can be positioned at any other location along the pollen collector device 100.

It is to be understood herein that in some examples, the pollen collector device 100 may not include some or all the above-described components, i.e., the handle 102, the reach extender 104, the LED(s) 143, and the solder cables. In such examples, the power controller 120 can be positioned in proximity to the pollen attractor 110 (for example, to constitute a single unit or arrangement), and the pollen attractor 110 can be configured to be detachably attachable, either directly or indirectly (such as via the power controller or any other connecting element), to a reach extender 104 (e.g., a rod or pole). Reach extender, in some embodiments, includes an elongated hollow through which power controller 120 is interconnected with pollen attractor 110.

In the example illustrated in FIGS. 1 and 24-2C, the pollen collector device 100 further comprises a floating grounding protrusion 140 electrically coupled to the power controller 120 to enable grounding of excess charge accumulated on the pollen collector device 100. For instance, during usage of the pollen collector device 100, electrons from surroundings of the pollination device can get accumulated on the pollen collector device 100 which results in reduced potential difference between the pollen attractor 110 and the power source. Thus, a net electric potential at the pollen attractor 110 is reduced thereby reducing the overall efficiency of the pollen attractor 110. This accumulated charge needs to be discharged from the device by grounding. The floating grounding component can be electrically coupled to the power controller 120 either directly or indirectly. In some examples, the floating grounding protrusion 140 can be electrically coupled to any other part of the pollen collector device 100 for discharging (or grounding) the excess accumulated charge.

As can be seen in FIGS. 24-2C, the grounding protrusion 140 is a floating grounding component, i.e., is carried along with the pollen collector device 100 and is not dependent on a fixed grounding element to discharge the accumulated charge to the ground. The floating grounding protrusion 140 includes a conductive portion 142, which can be electrically exposed to surroundings of the pollination device either permanently or when required, for example during generation of the electrostatic field by the pollen attractor 110.

During use of the pollen collector device 100 for attracting and collecting the pollen, the conductive portion 142 comes into contact with the plant and discharges the accumulated charge through the plant to the ground (earth). Accordingly, in the illustrated example, the floating grounding protrusion 140 is positioned in proximity to the pollen attractor 110 that is to be positioned over (or near/proximal to) the plants, so that the conductive portion 142 engages the plant during use of the device. In some examples, the floating grounding protrusion 140 can be positioned at any other location that the one illustrated herein while being able to engage the plant during use of the device. It is to be understood herein that the floating grounding protrusion 140 is positioned to be in contact with the plant and to be spaced apart from the user operating the device 100 to protect the user from electric shock from the floating grounding protrusion 140.

In the illustrated example, the floating grounding protrusion 140 has been shown to be formed as a protrusion 140 protruding from the top of the pollen attractor 110. In some examples, the protrusion 140 can be formed to protrude from any other location in proximity to the pollen attractor 110. In some examples, the protrusion 140 can be formed to be elastically resilient enough to bend upon engaging the plants. The protrusion 140 can be formed to be elastically resilient at least enough not to harm either the plant or the protrusion 140 in case it gets stuck in the plant.

In some examples, the floating grounding protrusion 140 can be formed in any other shape and/or size while being enabled to engage the plant without affecting the use of the device. In some examples, the floating grounding protrusion 140 can be formed as a frame corresponding to the pollen attractor 110.

It is to be understood herein that in some examples, the pollen collector device 100 may not include the floating grounding protrusion 140. In such examples, the excess charge accumulated on the device may not be discharged at all and the pollen attractor may still perform its intended function, for example, with a reduced efficiency, which can be compensated by other means such as by regulating the power from the power source.

In the illustrated example, the pollen attractor 110 includes an electrode surface 112, which can be used to collect the attracted pollen thereupon. In some examples, the pollen attractor 110 may not include the electrode surface 112, and can be formed as an empty frame or any other shape including ring shaped, polygonal frame, or even as a rod-shaped element configured to generate electrostatic field by virtue of the high voltage provided thereto for attracting pollen. In such examples, the pollen attractor 110 can include a pollination cartridge having a pollen adsorbing matrix for collecting the attracted pollen thereupon, for example, the pollination cartridge 10, as described in detail further herein below with reference to FIGS. 3A-3C. In such examples, the pollen collector device 100 can include pollination cartridge attachment elements configured for detachably attaching a pollination cartridge (for example, the pollination cartridge 10 described herein or a similar pollination cartridge) having a pollen adsorbing matrix configured for collecting the pollen thereupon.

In the illustrated example, the pollen collector device 100 has pollination cartridge attachment elements 106 positioned at the pollen attractor 110. A pollination cartridge, such the pollination cartridge 10, can be detachably attached to the pollination cartridge attachment elements 106, as described in detail further herein below with reference to FIGS. 3A-3C.

In some examples, the pollination cartridge attachment elements 106 can be any quick attachment and detachment elements including a hook-and-loop fastener, magnetized, snap connector, or any other adhesive or fixing/connecting/mounting elements for detachably connecting the pollination cartridge thereto.

The variation of pollen collector device 100 shown in FIGS. 2D-2E includes a combined handle 102 and power controller 120. Also shown are a variation of a floating grounding protrusion 140A, in which conductive portion 142A is implemented as a curved plate, positioned where it can be periodically connected to ground by brushing it against a leaf or other plant part. This operation can be performed to restore a full electrical potential relative to ground, before returning the device to floating voltage operation. Also shown is optional LED indicator 143, which illuminates to display status information. For example, LED indicator 143 is lit (e.g., lit orange or red) to confirm (and warn) that electrical fields are being generated as intended by charged pollen attractor 110, and/or lit to indicate exception conditions such as an insufficient voltage gradient being generated, a need to reset the floating ground level by coupling conductive portion 142, 142A to a ground voltage of the surroundings, and/or a low battery power condition.

In some examples, LED indicator 143 and/or another sensory indication is used to guide a device operator in performing a pollen exchange procedure. In some examples, one or more indications of operation of the pollination device are accessed by a controller, the indications processed for evaluation of one or more criteria of targeted device operation, and guidance provided to a device operator using user interface 144; e.g., LED indicator 143 or another indicating device.

For example, movement patterns of pollen collector device 100 are optionally sensed (e.g., using one or more on-device inertial state sensors). Such patterns can optionally include one or more of: sensing movements consistent with lowering the device for exchange of pollination cartridge 10, brushing the distal end of pollen collector device 100 over a plant, and accelerations consistent with lifting, lowering, and changing the angle of pollen collector device 100 to place charged pollen attractor 110 at a range of heights. Optionally geographical position of charged pollen attractor 110 is measured using a GPS receiver, and/or an inertial sensor. In some examples, these movement patterns are compared to targeted and/or non-targeted movement patterns, for example, using a machine learning product trained to distinguish such patterns, and/or using an algorithm configured to compare measured movement patterns to suitable templates and/or thresholds of movement. For example, a targeted movement pattern may include brushing motions within each of a plurality of height ranges, repeated for each lateral (geographical) translation of pollen collector device 100 to a different plant, and/or occurring between periods of lowering and/or deactivating charged pollen attractor 110 which are consistent with and/or indicative of the replacement of pollination cartridge 10.

In some examples, operation which is not consistent with targeted movement patterns results in activation of LED indicator 143, and/or another sensory indicator such as a tone, haptic indication (e.g., a vibrating device), and/or screen text, e.g., on a user interface 144 of power controller 120 and/or handle 102 (e.g., a user interface 144 comprising one or more buttons, knobs, sliders, touch screens, displays, and/or other sensory indicators). LED indicator 143 should also be considered as an element of remote user interface 144. It has a particular potential advantage for pollen collector device 100, by being placed along reach extender 104 at a position which a device operator is likely to have in view while also moving charged pollen attractor 110 and pollination cartridge 10 to perform pollen exchange operations. Optionally, LED indicator 143 is placed along reach extender 104, at least 1 m from handle 102, and/or within 10 cm of charged pollen attractor 110. Optionally, LED indicator 143 is enabled to communicate at least two distinct types of operating state indications to an operator, for example, one of the types described with specific reference to LED indicator 143.

Optionally, e.g., upon the satisfaction of targeted movement pattern conditions for a particular plant and/or pollination cartridge 10, sensory indicators are used to indicate that operations should proceed to the next plant and/or pollination cartridge 10. For example, satisfactory plant coverage and/or a full cartridge condition is indicated. Ground resetting using conductive portion 142 and/or 142A is optionally suggested to an operator on a cycle determined by use time, use activity, and/or a measured voltage and/or voltage change; for example, voltage and/or power requirement fluctuations induced as charged pollen attractor 110 and/or pollination cartridge 10 are moved in the vicinity of plants.

In some embodiments, time indications are used as the basis of the indications, criteria, and guidance. For example, the indications optionally comprise indications of the beginning and end of device activation; e.g., times at which a user activates and de-activates production of an electrostatic field. Optionally, indications comprise indications of times at which pollination cartridge 10 is replaced. Criteria optionally are based on timings; for example, how long charged pollen attractor 110 is activated, and/or how long a period of operation has elapsed since a recent event related to device operation, e.g., cumulative operation time since the last exchange of pollination cartridge 10. Optionally, criteria themselves include one or more time-dependent parameters; for example, times to begin and/or end device operation, e.g., selected based on estimated time periods during which pollen is ready for collection, and/or plants are ready to be pollinated. User interface indications are optionally selected as appropriate to warn the user, update the user, and/or draw the user's attention to conditions of sufficiency and/or insufficiency with respect to the device operation criteria.

Optionally, pollen collector device 100 is configured to perform imaging, e.g., using a camera, LIDAR, and/or other light sensor positioned at any suitable location on pollen collector device 100. Imaging is optionally used to detect plant contacts and/or plant proximity. Optionally, imaging is used to distinguish and/or provide indications of operational states such as contact or non-contact with plant parts, contact or non-contact with targeted plant parts (e.g., flowers in view, optionally evaluated according to color, size, and/or shape), and/or excessively heavy contact, e.g., heavy darkening due to foliage obscuring the camera. Optionally, imaging is used as part of a safety mechanism, e.g., to detect potentially human movement in proximity to charged pollen attractor 110 and shut it down if appropriate. Optionally, device imaging is used to survey a work site; e.g., an optical camera can be used to survey a plant and/or the ground around a plant, as a way of assessing operational quality (e.g., broken plant parts), for example as part of training device operators.

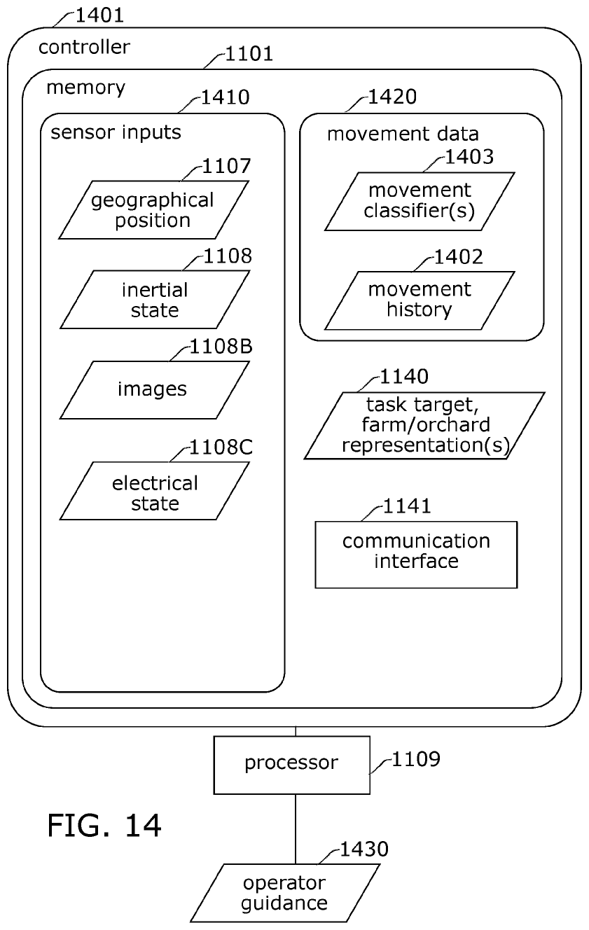

Reference is now made to FIG. 14, which is a block diagram schematically representing components, inputs, and other data structures of a controller 1401 for a hand-held pollen collector device 100, according to some examples of the presently disclosed subject matter.

Controller 1401 is optionally housed anywhere suitable in pollen collector device 100, for example, integrated into handle 102 and or arm-mounted power controller 120. Optionally, a portion of controller 1401 is implemented remotely, e.g., via wireless connection to a base station.

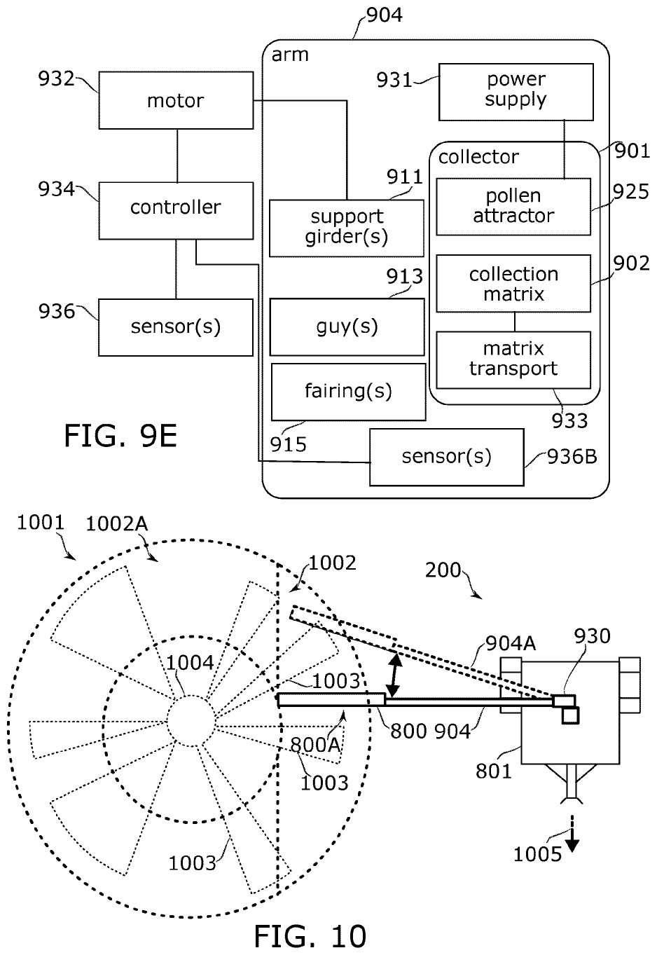

Components of controller 1401 corresponding to features of FIG. 1l are optionally implemented, e.g., also as described in relation to FIG. 11; changed as appropriate for the hand-held configuration of pollen collector device 100 (e.g. lacking the “arm”, but having other operating parameters relating to movement), and/or as next described.

In particular, memory 1101 optionally receives and/or stores sensor inputs 1410 for parameters such as geographical position 1107, inertial state 1108 and/or images 1108B, each obtained from appropriate corresponding sensors, e.g., as described in relation to FIG. 11 and/or FIGS. 2A-2E. Optionally, electrical state 1108C is sensed as one or more inputs; this may include, for example, one or more of: the on/off state of electrostatic field generation; an indication of actual voltage of an electrostatic field being generated; a charge state of a power source; an indication of a fault such as a short, fuse and/or breaker opening, and/or high temperature condition; contact and/or Hall effect sensing indicative of the proper placement of a pollination cartridge 10, fluctuations in a static electrical field generated through charged pollen attractor 110 (e.g., fluctuations induced by interaction with the environment); or another electrical state indication. The availability of particular types of sensor inputs 1410 should be taken as an indication that appropriate sensor types are correspondingly provided to a pollen collector device 100; for example, mounted with handle 102, power controller 120, reach extender 104, and/or on the distal end of charged pollen attractor 110 on or near charged pollen attractor 110.

Movement data 1420 optionally includes data and/or instructions defining one or more movement classifiers 1403, which operate, for example, as described in relation to FIGS. 2A-2E and device operation-based guidance. Optionally, a movement history 1402 is stored, e.g., based on sensor inputs 1410, and used by processor 1109 when it applies the movement classifiers 1403. The task target and/or farm/orchard representations of block 1140 are optionally received via communication interface 1141, and/or entered or selected, e.g., by a user interface of pollen collector device 100.

Processor 1109, in some examples, uses any appropriate data, e.g., as described in relation to memory 1101, to perform calculations leading to the production of indications for operator guidance 1430. This can include indications provided to any display or other indicator described in relation to LED indicator 143 and/or user interface 144 more generally. Available operator guidance 1430 optionally includes indications for one or more states of pollen collector device 100 itself (e.g., its electrical and/or operating states), and/or indications evaluating operation of pollen collector device 100, e.g., based on movement patterns, imaging, and/or on another sensed basis.

Reference is now made to FIGS. 3A-3C, which schematically illustrate the pollination cartridge 10, according to some examples of the presently disclosed subject matter. The pollination cartridge 10 is detachably attachable to a pollination device, such as the pollen collector device 100 and the pollinating device 50. The pollination cartridge 10 has the pollen adsorbing matrix 12 configured to collect the pollen 401, store the pollen, and/or transfer pollen to perform pollination. The pollination cartridge 10 comprises attachment elements 14 for detachably attaching the pollination cartridge to the pollination devices. For instance, the attachment elements 14 can be detachably attached to the pollination cartridge attachment elements 106 of the pollen collector device 100 for attaching the pollination cartridge 10 to the pollen collector device 100, and to the connectors 58 of the pollinating device 50 for attaching the pollination cartridge 10 to the pollinating device 50.

In the illustrated example, the attachment elements 14 are disposed at a periphery P of the pollination cartridge 10. In some examples, the attachment elements can be disposed at any other location at the pollination cartridge 10 for attaching the pollination cartridge 10 to corresponding connectors of the pollination device. It is to be understood herein that most of the attachment elements 14 are disposed at the periphery P not to interfere with the pollen collecting/storing/releasing capabilities of the pollen adsorbing matrix 12.

The pollination cartridge 10 can be attached to the pollen collector device 100 while being positioned to engage the pollen attracted by the pollen attractor 110 and collect the pollen on the pollen adsorbing matrix 12. In some examples, the pollen adsorbing matrix can 12 include a dielectric material, in a sheet form such as a membrane or textile (e.g., a woven fabric of polymer yarns or threads) which can include an electrical insulating material. In some examples, the pollen adsorbing matrix 12 comprises, for example, PVC, nylon, polyester, polyethylene, PTFE, or another polymer. Optionally, the dielectric material comprises a first polymer selected for a structural property such as toughness or resistance to environmental exposure, and a second polymer (e.g., PVC), used as a coating, selected according to its properties of electrostatic attraction and/or pollen grain adherence, optionally including reversibility of pollen grain adherence.

The dielectric material gets charged by the electrostatic field generated by the pollen attractor 110 and can hold the pollen more effectively. The pollen sticks to the pollen adsorbing matrix 12 and keep sticking even after the pollination cartridge 10 is detached from the pollen collector device 100. The dielectric material might lose the charge once the pollination cartridge 10 is detached from the pollen collector device 100, but particularly in the absence of further mechanical disturbance, the pollen remains attached to the pollen adsorbing matrix 12. The pollination cartridge 10 can then be used to store the pollen and to transfer the pollen to a desired location without the need for a separate container for the purpose. The thickness of the pollen adsorbing matrix 12 can range between 0.1 mm to 50 mm, In some examples, the thickness is in a range suitable for a flexible solid sheet or woven sheet (textile), e.g., between about 0.1 mm to about 5 mm, or about 0.5 mm to about 2 mm. Using a thin substrate pollen adsorbing matrix 12 potentially reduces space requirements associated with pollen storage and/or transfer.

Fabric examples of pollen adsorbing matrix 12 may comprise a woven structure, optionally 300 Denier (300 D), 420 Denier (420 D), or another yarn density.

Comparison tests of 420 D to 300 D fabrics noted a potentially increased ease with which pollen grains can be dislodged from a coarser-woven (e.g., 300 D) fabric, optionally coupled with relatively smoother yarns (e.g., yarn with larger and/or more uniformly arranged fibers). For example, a given thickness of the yarn may have 10 or fewer fibers, 5 or fewer fibers, or 2 or fewer fibers. have less than

Finer weave structure can potentially increase the pollen-retaining capability of the woven structure considerably. The fiber structure of the yarn or thread also potentially contributes; e.g., using a looser and/or finer yarn fiber structure potentially creates more fine protrusions to which pollen 401 can attach. While this is a potential advantage for resisting inadvertent pollen loss, it may conversely result in over-retention during pollination itself. Accordingly, it is alternatively a potential advantage to provide a smoother fabric structure, e.g., using more tightly wound yarns and/or fewer individual fibers, at least when inadvertent loss of pollen during collection (e.g., due to “wiping” by foliage) is sufficiently controlled.

Additionally or alternatively, edges in the fine structure of a fabric potentially generate regions of concentration of the electrostatic field. This potentially increases collection yield, although it is also potentially associated with greater and/or tighter binding of attracted pollen 401. It should be understood that features of the composition and/or structure of pollen adsorbing matrix 12 optionally apply to pollen adsorbing matrix 902, 222 as described, e.g., in relation to FIGS. 8A-8B, 9A-9E, and/or 12A-13.

The dielectric strength of the dielectric material of the pollen adsorbing matrix 12 can range between 1 kV/mm and 1000 kV/mm. In some examples, a ratio between the dielectric strength and the thickness of the pollen adsorbing matrix 12 ranges between 0.3 kV/mm² and 800 kV/mm² and can be selected to achieve the most effective collection of the pollen thereupon.

Dimensions of pollination cartridge 10 are, for example, about 350 mm×350 mm, optionally with two or more rounded corners having a radius about 75 mm (e.g., ±10% and/or ±5-10 mm). In a particular example, the dimensions are 353.75 mm×353.75 mm, with a corner radius of 76.87 mm.

The pollen adsorbing matrix 12 is configured to release the collected pollen as appropriate, for example by wiping off the pollen therefrom upon contact with parts of plant flowers receptive to pollination. For instance, the pollen adsorbing matrix 12 can be used to collect the pollen from a source plant (or male parts of a plant) and can be used to deploy the collected pollen at the target plant (or female parts of the plant) thereby assisting in artificial pollination, e.g. by wiping pollen across receptive flower elements such as flower stamens. For releasing the pollen from the pollen adsorbing matrix 12, the pollination cartridge 10 can be attached to the pollinating device 50.

It is to be understood herein that although in the illustrated example, the pollination cartridge 10 has been shown as having a frame-like shape corresponding to that of the pollen attractor 110 and the pollination cartridge support 56, the pollination cartridge 10 and/or pollen adsorbing matrix 12 can have any other shape and size to effectively collect, store, and deploy the pollen. In some examples, the pollen adsorbing matrix can be formed to be elastically resilient enough to reversibly bend upon engaging the plant. The pollination cartridge 10 and/or pollen adsorbing matrix 12 can be formed to be elastically resilient at least enough not to harm either the plant or the pollination cartridge/surface in case it gets stuck in the plant or when it engages the plant. Herein, the distal end including pollination cartridge support 56 is optionally referred to as a pollen exchanger, with or without an installed pollination cartridge 10.

### Vehicle-Drawn Pollen Exchange Devices

### Fixed-Matrix Pollen Exchange Devices

Reference is now made to FIGS. 8A-8B, which illustrate a vehicle-drawn artificial pollination system 200, according to some examples of the presently disclosed subject matter. FIG. 8A schematically illustrates the system 200, and FIG. 8B is a photographic image of the system 200 drawn behind a tractor 840, according to some examples of the presently disclosed subject matter. The pollination system 200 is configured to be mounted on a carrier, for example, carrier 801, and drawn through a field/orchard to perform the artificial pollination. In the example shown, carrier 801 is implemented as a wheeled trailer, comprising base 802 and tire/wheel assemblies 803, along the options of spare tire/wheel assembly 803A, storage enclosure 804, tow hook 805, tow bar 806, and parking jack 807. Optionally, carrier 801 is any vehicle configured to carry the pollination system 200 in the field/orchard along a pollination path alongside plants (that is, a desired or dedicated path in the field alongside plants) to enable operation of system 200 to assist in pollen collection and/or plant pollinations. In some examples, artificial pollination system 200 is provided with a user interface 144, which is optionally incorporated into storage enclosure 804, and or otherwise placed for control and/or monitoring of artificial pollination system 200. For example, artificial pollination system 200 optionally is configurable to communicate with a hand-held computing device (e.g., a cellular phone), and/or another device which can be mounted in view of an operator of tractor 840.

Mounted on carrier 801, the pollination system 200 comprises mast 202. Mast 202 is an example of a support structure configured to be mounted to the carrier 801, and from which arms 804A-804C are supported. As depicted, three mechanical arms 204A-204C are connected to the mast 202. Each is positioned so that its respective pollen exchanger 800 is positioned on a lateral side of a base 802 of carrier 801, with a plant-engaging surface 800A facing into the direction of travel, and positioned for pollen collection and/or pollination. In some embodiments, plant-engaging surface 800A is provided by an area of pollen adsorbing matrix 902, e.g., comprising about 50 cm×50 cm. Optionally, the covered area is larger or smaller; e.g., with any suitable linear dimension between about 35 cm and 200 cm, e.g., about 100 cm×100 cm. In some examples, the vertical dimension is taller, e.g., up to 2 m, 3 m, or more.

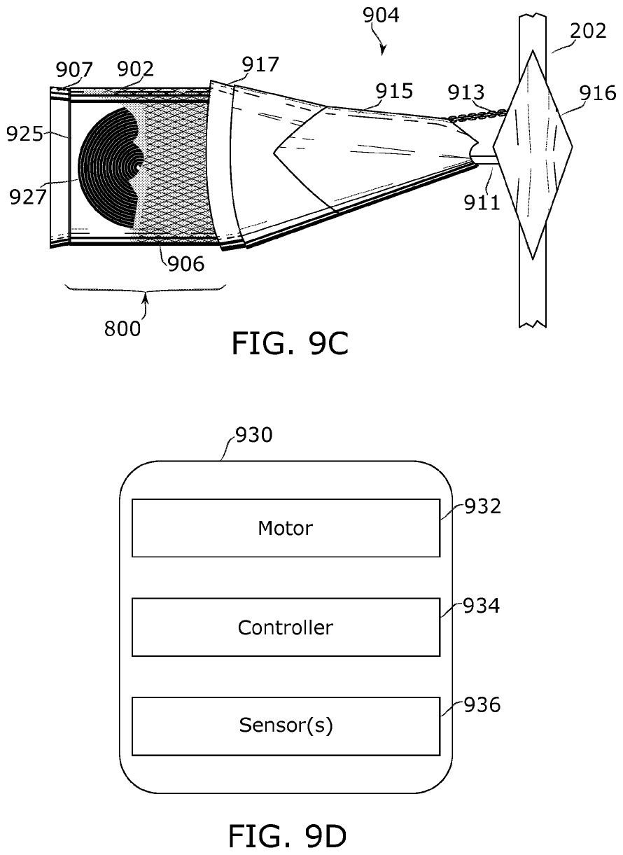

The mechanical arms 204A-204C are positioned so that they cover different pollen producing and/or receiving zones of a targeted plant such as a tree, e.g., as described in relation to FIG. 10. In the example depicted, all three arms extend to the same side (the right, indicated by R in axis rose 820), but it should be understood that either and/or both sides are optionally used (e.g., optionally including the left side indicated by L in axis rose 820), and that heights are optionally adapted as appropriate to the height of plants from which pollen is collected and/or to which pollen is transferred.

It is also to be understood herein that although in the illustrated examples, three mechanical arms are shown, the system 200 can include a lesser or greater number of mechanical arms connected to the mast 202. The mechanical arms have been described below with reference generally made to the mechanical arm 204A. It is to be understood herein that the below description applies to one or more of the other mechanical arms 204B and 204C as well.

Mast 202 includes an optional folding capability, implemented by dividing it into mast base section 202A and mast mounting section 202B. Locking brace 810 of mast mounting section 202B engages with mast base section 202A using locking pin with cotter pin 812. To stow mast mounting section 202B, the cotter pin is removed from the locking pin, and the locking pin removed, allowing the top portion of mast mounting section 202B to swivel down and backward on swivel 809 (to which it is hinged) with its weight at least partially cushioned by pneumatic support 809. Locking pin with cotter pin 813 may then be used to lock mast mounting section 202B to receiving bracket 814 of stow support 811. Optionally, mechanical arms 204A-204C are further partially disassembled for transport, e.g., by removal of the pollen attractors 210, which storage enclosure 804 is optionally sized and otherwise configured (e.g. with internal support elements) to house.

The mechanical arm 204A has an arm proximal end 204P connected to the mast 202 and an opposite arm distal end 204D. Optionally, at least a portion of the weight of mechanical arm 204A is supported by guy 817, extending from mast 202 to a location along spar 911. The mechanical arm 204A is connected to the mast 202 by a hinged connector 206 so as to provide the mechanical arm 204A, or at least the arm distal end 204D, a first degree of freedom which allows movement largely along a first axis, depicted at axis rose 820 in FIG. 8A between directions F and B. The first axis is generally oriented along the pollination path.

It is to be understood herein although the directions F and B have been shown to be straight to depict the direction of degree of freedom, the path followed by the arm distal end 204D can be along a curved path 831 between more forward and more rearward positions, also involving lateral travel along the axis between R and L; for example, by virtue of pivoting of the mechanical arm 804A. For instance, the pollination path is the path along which carrier 801 travels while carrying the pollination system 200, and the first direction extends along the pollination path.

The first degree of freedom is constrained (or restrained) to prevent complete bending of the mechanical arm in any direction. For instance, the first degree of freedom allows the arm distal end 204D to move in the first direction at least between a forward position and a backward position along the pollination path. The forward and backward positions are to be understood with respect to the direction of movement of the carrier 801. The mechanical arm 204A is biased to be in a normal (resting) position with respect to the mast 202; for example, a position for which the arm distal end is in the forward position. Upon engaging with a plant portion indirectly or directly (for example during forward movement of the carrier along the pollination path), the arm distal end 204D is urged in backward direction B by virtue of the first degree of freedom. By this motion, arm mechanical arm 204A also assumes a raked (back-deflected) angle and/or is laterally retracted. This potentially reduces the amount of deflection which is reciprocally exerted on plant branches, allowing them to pass by, after contact, without having to bend to the full extent than a non-deflecting arm could impose. This has the potential advantage for reducing damage in particular to the flowers 400 and/or their inflorescences 501 which brush against pollination cartridge 220 and pollen adsorbing matrix 222. For relatively lightly constructed examples of mechanical arm 204A, it is potentially sufficient to implement restorative forces for mechanical arm 204A using spring 818, attached to and extending between a location along spar 911 of arm 204A, and arm proximal end 204P. The depiction includes optional partial enclosure of spring 818 by spring housing 819.

Once past the plant (and relieved of the forces plant branches exert), the arm distal end 204D moves in forward direction F by virtue of the bias applied thereto (e.g., via spring 818), and returns automatically to the forward position. In some examples, the mechanical arm 204A can bend in the backward direction up to a backward position making a maximum raked angle ranging between 0 to 90 degrees with respect to the normal forward-biased position (which in the illustrated example is the forward position shown in FIGS. 8A-8B). Thus, the normal forward-biased position and the backward position define a range of motion constraint on the first degree of freedom. In some examples, mechanical arm 204A is configured to bend in the forward direction further forward from the normal forward-biased position (the one shown in FIGS. 8A-8B) by an angle ranging between 0 to 90 degrees with respect to the normal biased position, thereby defining a forward position further forward from the normal forward-biased position. Typically, however, the most forward-biased position of mechanical arm 204A is selected to be approximately perpendicular to the direction of travel, so that when branches are encountered, further backward motion of mechanical arm 204A leads to a reduced extent of lateral extension, without much if any initial increase in lateral extension (e.g., less than 5% increase to a full extent of lateral extension).

In some examples, additionally or alternatively to the hinged connector 206 providing the first degree of freedom, the mechanical arm 204A can be made to be elastically resilient enough to bend upon engaging, directly or indirectly, the plants. This optionally comprises, for example, one or both of bending along spar 911, and bending of pollen exchanger 800.

It is to be understood herein that the maintaining and restorative forces acting on the mechanical arm 204A are selected to enable raking and/or reversible bending at least to such an extent that the mechanical arm 204A does not harm the plant (and in particular, the inflorescences 501 of the plant) during operation, and/or so that such harm as occurs is infrequent and minimal in extent. For example, less than 10% of inflorescences contacted are left broken; optionally less than 5%. The maintaining and restorative forces acting on the mechanical arm 204A can be selected on the basis of the density of the plants, the speed at which the carrier is intended to be moved, the strength of the branches of the plant, and length of the mechanical arm so as to ensure that during the operation of the system, neither undue plant damage occurs, nor damage to the mechanical arm 204A. In some examples, a force equivalent to about 4 Newtons of force (that is, equivalent to the weight of about 400 grams) applied at the most lateral edges of an arm mechanical arm 204A (e.g., the lateral edge of pollen exchanger 800) is sufficient to induce deflection from the forward-biased position of arm mechanical arm 204A. In some examples, a different restorative force is used, e.g., a force in the range of about 2.5 N to 15 N.

Restorative forces may increase somewhat as a function of increasing deflection angle; e.g., due to extension of spring 818, and/or development of other restorative spring forces as a result of flexing along mechanical arm 204A. Optionally, however, anchor positions of spring 818 are selected so that they do not move to substantially more distant positions upon deflection of arm mechanical arm 204A from its forward-biased angle. This allows selecting restorative force substantially using pre-tensioning. It is noted that with increasing mass of arm mechanical arm 204A (and in particular, increasing angular inertia), a greater share of the forces exerted on arm mechanical arm 204A may be due to arbitrary rocking of carrier 801 as it travels. This potentially leads to a requirement for active control of the deflection angle of arm mechanical arm 204A; for example, as described in relation to mechanical arm 904 of FIGS. 9A-9E, which is optionally provided in place of any one or more of mechanical arms 204A-204C of FIGS. 8A-8B.

In some examples, the hinged connector 206 further enables a second degree of freedom in a second direction, depicted in FIG. 8A by arrows U and D of axis rose 820, vertically perpendicular to the pollination path as well as to the mechanical arm 204A. It is to be understood herein although the arrows U and D have been shown to be straight to depict the direction of degree of freedom, the path followed by the arm distal end can be along a curved path, for example, by virtue of pivoting of the mechanical arm. For instance, the second direction includes a vertical direction having an upward direction component U and a downward direction component D, optionally with associated movement along the axis extending between R and L. The second degree of freedom is constrained (or restrained) to prevent complete bending of the mechanical arm in any direction. For instance, the second degree of freedom allows the arm distal end 204D to move in the second direction at least between an upward position and a downward position from a resting position shown in FIGS. 8A and 8B. The upward and downward positions are to be understood with respect to a vertical plane including the mechanical arm 204A. The second degree of freedom enables the movement of the mechanical arm 204A whenever required to move the mechanical arm 204A through the plants. For instance, if the mechanical arm 204A, or a component attached thereto, gets stuck in the branches, the second degree of freedom allows the user to manoeuvre the mechanical arm 204A out of the branches. In some examples, the mechanical arm 204A can bend in the upward direction U up to an upward position making an angle ranging between 0 to 90 degrees with respect to the normal resting position and/or in the downward direction D up to a downward position making an angle ranging between 0 to 90 degrees with respect to the normal resting position. Thus, the upward position and the downward position define the constraint of the second degree of freedom. In some examples, the upward or the downward position is same as the normal resting position. Movement optionally comprises detaching and/or loosening of one or more guys 817. For example, a crank and/or motor (not shown) is operated to loosen guy 817 and allow mechanical arm 204A to droop downward. Optionally, the second degree of freedom is operated as part of stowing artificial pollination system 200 for travel; e.g., to fold mechanical arms 204A-204C inward against mast mounting section 202B while stowed.

In some examples, in addition to or in alternative to the hinged connector 206 providing the second degree of freedom, the mechanical arm 204A (e.g., a portion of spar 911) can be made to be elastically resilient to provide the second degree of freedom.

In some examples, the pollination system 200 can include one or more power controllers 931 (optionally housed, e.g., in storage enclosure 804), corresponding to the power controller 120 described above with respect to the pollen collector device 100, configured for electrically connecting thereto a power source. It is to be understood herein that descriptions provided above with respect to the power controller 120 applies to the power controller 931 of the pollination system 200 as well, changed as appropriate. The pollination system 200 can further include an electrical interface (not shown) connected to the arm distal end 204D and configured for electrically coupling the power controller 931 and a pollen attractor; for example, corresponding to the pollen attractor 110 described above with respect to the pollen collector device 100, when connected at the arm distal end 204D. Control and/or power arrangements described in relation to the example of FIGS. 9A-9E optionally apply as appropriate; e.g., an arm-mounted power controller 120 and/or a associated power supply, whether supplying high voltage (e.g., of 1 kV or more, e.g., as described also in relation to high voltage and pollen collector device 100) to just one arm, or a plurality of arms. Optionally, control and/or power arrangements are housed by storage enclosure 804, and/or positioned with mast 202 and/or arms 204A-204C as appropriate.

Pollen exchanger 800 comprises pollen attractor 210, which is provided with an electrical charge created by the supplied high voltage in order to attract pollen. It is to be understood herein that descriptions provided above with respect to the pollen attractor 110 apply to pollen attractor 210 as well, changed as appropriate. For example, pollen attractor 210 may be larger and powered from a bulkier power supply. In the example, shown, pollen attractor 210 is provided with an electrode arrangement comprising an electrically conductive pathway which passes back and forth over the flat extent of pollen attractor 210 in a square wave pattern. It should be understood that other electrode arrangements are optionally provided, e.g., the spiral arrangement of the example of FIG. 9C.

The electrical interface establishes an electrical connection between the power source and the pollen attractor 210 (when connected to the arm distal end 204D) to enable the pollen attractor 210 to generate an electrostatic field to attract the pollen. In such examples, the mechanical arm 204A at least partially defines a connection path for providing the electrical coupling between the arm distal end 204D and the power controller 931, for example by wiring. Thus, when the pollen attractor 210 is connected to the arm distal end 204D, the electrical connection between power controller 931 and the pollen attractor 210 can be established via the electrical interface and the wiring.

In some examples, the power controller 931 can constitute a part of the pollen attractor 210. For instance, the pollen attractor 210 can be stand alone component including the power controller 931 as well as the power source and all the required circuitry (electrical interface, step up converter/transformer, etc.) to generate the electrostatic field. In such examples, the pollination system can include the pollen attractor 210, and the pollen attractor 210 can be configured for detachably or fixedly connected to the arm distal end 204D. In some examples, the pollen attractor 210 can include the power controller 931 and can be configured to be electrically couplable to an external power source. For example, a housing power controller 931 is optionally provided immediately adjacent to pollen attractor 210, for example as described in relation to pollen attractor 925 of FIGS. 9A-9E.

It is to be understood that descriptions provided hereinabove with respect to the power controller 120, pollen attractor 110, and power source with reference to FIGS. 1 and 24-2C apply to corresponding components of the pollination system 200 as well, changed as appropriate to the scale and/or other interconnected features of artificial pollination system 200. Descriptions of these features are not repeated here for the sake of conciseness of the present description.

Pollination system 200, in some embodiments, further comprises a pollination cartridge 220 connected to the pollen attractor 210, for example as depicted in FIG. 8A (gray areas). It is to be understood that pollination cartridge 220 corresponds to the pollination cartridge 10; and that descriptions provided above with respect to the pollination cartridge 10 apply to the pollination cartridge 220 as well, changed as appropriate to the scale and other interconnected features of artificial pollination system 200. For instance, the pollination cartridge 220 comprises a pollen adsorbing matrix 222 configured to adsorb pollen from plants with which it engages. The pollen adsorbing matrix 222 physically contacts (engages) and/or closely approaches the plants during movement of the pollination system 200 along the pollination path. Voltages applied by pollen attractors 210 positioned behind pollen adsorbing matrix 222 encourage pollen to transfer to pollen adsorbing matrix 222. The pollen adsorbing matrix 222 and supporting pollen exchanger 800, in some examples, is elastically resilient enough to reversibly bend, e.g., as shown by arrow 832, in a direction perpendicular to the pollen adsorbing matrix 222, i.e., the direction along the pollination path.

It is to be understood herein that the elastic resilience of the pollen adsorbing matrix 222 is so selected to enable reversible bending of the pollen adsorbing matrix 222, e.g., to an extent such that the pollen adsorbing matrix 222 does not harm the plant during operation, and/or so that such harm as occurs is infrequent and/or minimal in extent. For example, less than 10% of inflorescences 501 contacted are left broken; optionally less than 5%. Bending forces are optionally selected, e.g., in the range of 2.5 N to 15 N; for example as described in relation to mechanical arm 204A overall. The resilience of the pollen adsorbing matrix 222 and/or supporting pollen exchanger 800 can be selected on the basis of the density of the plants, the speed at which the carrier is intended to be moved, the strength of the branches of the plant, size of the pollen adsorbing matrix 222 so as to ensure that during the operation of the system, neither the plants nor the pollen adsorbing matrix 222 is damaged. To further prevent the pollen adsorbing matrix 222 from damaging the plants, the pollen adsorbing matrix 222 is made as a smooth surface to physically contact or engage the plants smoothly and the pollination cartridge is formed with smooth curved corners. Optionally, a net or other perforated protection is placed in front of pollen adsorbing matrix 222 to protect it, for example as described in relation to FIGS. 9A-9E.

It is to be further understood herein that the pollen adsorbing matrix 222 corresponds to the pollen adsorbing matrix 12. Descriptions provided above with respect to the pollen adsorbing matrix 12 apply to the pollen adsorbing matrix 222 as well, changed as appropriate for scale and/or for appropriate interoperation with associated components. For instance, the pollen adsorbing matrix 12 is configured to collect, store, and deploy the pollen in a similar manner as described above with respect to the pollen adsorbing matrix 12.

In some examples, the pollination system 200 omits pollen attractor 210. The pollination cartridges 220 can be connected (detachably or fixedly) to pollen exchanger 800 either directly or indirectly (via an additional element). In such examples, the pollen adsorbing matrix 222 can be configured to collect and/or release the pollen by contacting (engaging) the plants.

### Transporting-Matrix Pollen Exchange Devices

Reference is now made to FIGS. 9A-9C, which schematically illustrate a mechanical arm 904 used in an artificial pollination system 200, according to some examples of the presently disclosed subject matter. Reference is also made to FIG. 9D, which is a block diagram schematically illustrating enclosed electronics of the mechanical arm 904, according to some examples of the presently disclosed subject matter. Further reference is made to FIG. 9E, which schematically illustrates the mechanical arm 904 as a block diagram, according to some examples of the presently disclosed subject matter.

In the example shown, pollen exchanger 800 comprises a transporting (in the example shown, scrolling) sheet of pollen adsorbing matrix 902, which corresponds generally in its use, features, structure, and other properties to those described in relation to pollen adsorbing matrix 12 and/or pollen adsorbing matrix 222, adjusted as appropriate in view of the scale, interconnecting components, and/or capabilities described in relation to pollen adsorbing matrix 902 more specifically. For example, pollen adsorbing matrix 902 optionally comprise include a dielectric material in a sheet form such as a membrane or textile (e.g., a woven fabric of polymer yarns or threads, optionally air-textured yards or threads). The dielectric material optionally comprises a polymer material, for example, PVC, nylon, polyester, polyethylene, PTFE, or another polymer. Optionally, the dielectric material comprises a first polymer selected for a structural property such as toughness or resistance to environmental exposure, and a second polymer (e.g., PVC), used as a coating. The coating is selected, for example, according to its properties of electrostatic attraction and/or pollen grain adherence; optionally including reversibility of pollen grain adherence.

Pollen collector 901 may be considered more generally as implementing a pollen exchanger 800; e.g., it can collect pollen while pollen attractor 925 is operating with a supplied high voltage to attract pollen to pollen adsorbing matrix 902; or perform pollination when pollen adsorbing matrix 902 is dragged across plants to distribute the collected pollen (generally without activation of pollen attractor 925).

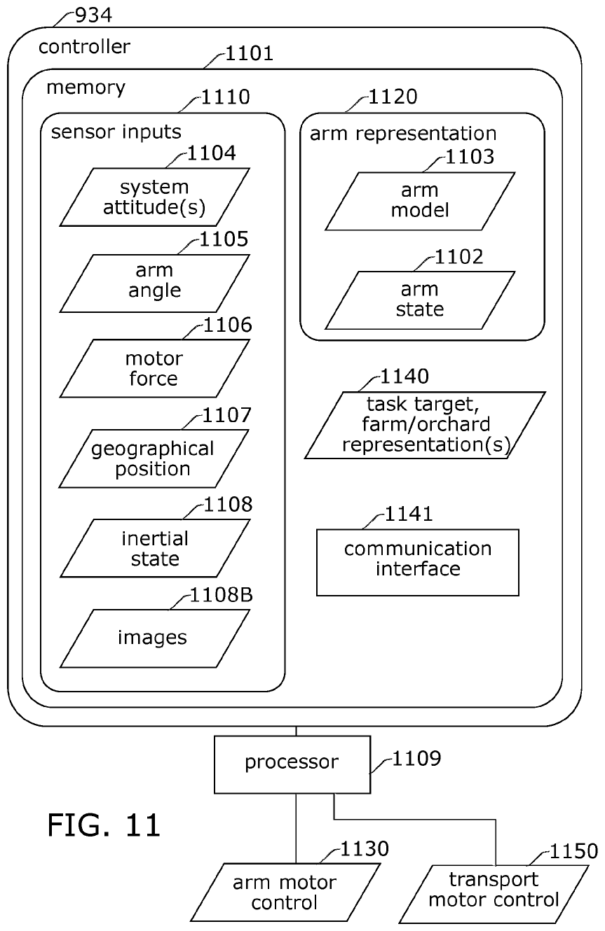

Pollen adsorbing matrix 902, in the example shown, comprises a long strip of dielectric material (e.g., a woven textile, extruded sheet, or other flat flexible structure) which is wound between rollers 920A, 920B, and passed between them according to the operations of motors 926, which are controlled to turn the rollers 920A, 920B in synchrony, e.g., as described in relation to FIG. 12A-12B. Optionally one or both of rollers 920A, 920B (or a portion thereof) is removable, e.g., allowing pollen adsorbing matrix 902 to be installed in a cartridge form comprising the removal roller portions. What remains in place may be referred to as a roller mounting location.

Scrolling or other matrix transport allows the adsorbing surface of pollen adsorbing matrix 902 to be refreshed periodically and/or continuously as artificial pollination system 200 advances along a row of plants. This provides a potential advantage by protecting already-collected pollen from being scraped and/or knocked off of pollen adsorbing matrix 902 by later plant contacts. Furthermore, collected pollen is, in effect, protected by storage within the coil of the roll. This potentially allows use of a pollen adsorbing matrix 902 which is less pollen-adherent; and this potentially increases pollen transfer efficiency during pollination. In some embodiments, a cartridge comprising pollen adsorbing matrix 902 also comprises one or more hollow cores (e.g., cylinders) around which the textile is wound. Optionally, one or both cores are detachable. Optionally, the cores are provided separately from the pollen adsorbing matrix 902 itself. In some preferred examples, pollen adsorbing matrix 902 is textured, the texture features being of a size large enough (e.g., rough enough) to engage with shapes and/or surface features of pollen grains, but small enough to avoid engulfing the pollen grains into the depths of the texture where they are potentially inaccessible to contact with flower parts receptive to pollination such as stamens. Adsorption is preferably reversible by contact, e.g., light brushing, particularly brushing by plant flower parts receptive to pollination such as flower stamens.

Mast 202 is optionally configured as described in relation to FIGS. 8A-8B, and optionally is replaced by any other suitable support structure. Optional fairings 915, 916 are provided to reduce instances of snagging on obstructions (e.g., the plants themselves) during operations to collect pollen and/or pollinate. Optional insulator 917 assists in resisting breakdown of electrical fields generated by pollen attractor 925, e.g., by electrical arcing to metal elements of mechanical arm 904. High voltage charging of pollen attractor 925 to establish a pollen-attracting electrostatic field (e.g., as described in relation to charged pollen attractor 110) is optionally powered through power controller 931. Optionally, charging power is provided from a power controller in another location, e.g., located within a storage enclosure 804, e.g., as described in relation to FIGS. 8A-8B. FIG. 9C shows a partial cutaway view of pollen attractor 925; a view which exposes a spirally arranged coil 927 normally embedded within an insulating overlay of pollen attractor 925. The coiled geometry provides a potential advantage for establishing an evenly distributed electrical field in front of pollen adsorbing matrix 902.

One or more guides 907 are optionally provided to guide pollen adsorbing matrix 902 as it passes between rollers 920A, 920B, and over the front-facing surface of pollen attractor 925. Optionally, protective net 906 extends in turn over the front-facing surface of pollen adsorbing matrix 902. Net 906 is optionally provided with apertures large enough to admit flowers 400 and/or portions of inflorescences 501, but small enough to prevent most ingress of abrading twigs and branches from plants being contacted. This provides a potential advantage for reducing wear such as tearing on pollen adsorbing matrix 902.

In some examples (e.g., as shown), main structural support of mechanical arm 904 comprises a spar 911 (to a distal end of which pollen exchanger 800 is mounted), supported by one or more guys 913, anchored as appropriate (e.g., to mast 202) to support the weight of the mechanical arm 904.

The combined mass of the elements of pollen exchanger 800 potentially creates a swinging moment of inertia which is relatively large compared to targeted levels of force which help mitigate plant damage due to contact with pollen exchanger 800 as it is advanced past a plant with which it engages. The targeted forces remain, e.g., within a range of about 2.5 N to 15 N; e.g., 4 N, and/or as described in relation to FIGS. 8A-8B.

In the example shown, accordingly, and in other examples as appropriate, sensing and active control are used to help maintain plant contact forces within a targeted range. Enclosure 930 (shown, e.g., in FIG. 9B, and diagrammed in FIG. 9D) optionally contains one or more of motor 932, controller 934, and sensors 936.

Motor 932, in some examples, comprises an electrical motor with sufficient power and torque to drive mechanical arm 904 into its forward-biased position, at least against its own weight (a potentially variable factor) plus the target force exerted on plants at the laterally outermost locations of mechanical arm 904. Potentially, requirements for torque and power are higher for movement in the reverse direction, in which case these set the minimum requirements.

Any of controller 934 and one or more sensors 936 are optionally housed on arm mechanical arm 904; e.g., in housing 930 as shown, and/or at other locations on carrier 801, and/or on structural components of an artificial pollination system 200. Control and sensing are further described, for example, in relation to FIG. 11. it should be understood that the elements of FIG. 9D are optionally provided to any artificial pollination system 200; e.g., a system using stationary cartridges as described in relation to FIGS. 8A-8B.

In block form, FIG. 9E illustrates elements described in relation to mechanical arm 904. Also included are optional sensors 936B, which may be provided in locations anywhere along mechanical arm 904, and used, for example, as described in relation to FIG. 11. The transport role of rollers 920A, 920B and motor(s) 926 is optionally provided by any suitable arrangement providing matrix transport 933, which serves at least the functions of positioning a first region of pollen adsorbing matrix 902 where it provides a plant-engaging surface 800A of pollen exchanger 800, and then refreshing plant-engaging surface 800A by moving a second region of pollen adsorbing matrix 902 into place to become all or part of plant-engaging surface 800A. It should be understood that the plant-engaging may involve physical contact and/or engagement via influences of an electrostatic field, e.g., as generated by pollen attractor 925.

The roller-based (scrolling) implementation of, e.g., FIGS. 9A-9E should be understood as an example. Optionally, for example, pollen adsorbing matrix 902 is provided in the form of a continuous loop (e.g., a belt). Matrix transport 933, accordingly, is optionally provided as motorized transport for this loop, which is optionally twisted and/or looped as appropriate in order to reach a targeted overall length. In some examples, pollen adsorbing matrix 902 is folded, e.g., folded in a storage box from and/or to which folds of pollen adsorbing matrix 902 are extracted and/or stored. Matrix transport 933, accordingly, is optionally implemented as a drag feeder; e.g., belts and/or sprockets which engage with pollen adsorbing matrix 902 to encourage its advance across pollen exchanger 800 and refresh plant-engaging surface 800A.

Transport 933, in some examples, moves (transports) pollen adsorbing matrix 902 across pollen exchanger 800, thereby refreshing a plant-engaging surface 800A of pollen exchanger 800 by exposing new regions of pollen adsorbing matrix 902 to plant contacts. In some examples, the movement is sufficient to result in at least one full replacement of an area of pollen adsorbing matrix 902 originally exposed as the plant-engaging surface 800A with a replacement area of pollen adsorbing matrix 902 during operations to collect pollen from flowers and/or pollinate flowers. In some embodiments, pollen adsorbing matrix 902 is a sheet with at least 5 times the area of the plant-engaging surface 800A, at least 10 times the area, at least 100 times the area, or another multiple of the plant-engaging surface 800A area of the pollen exchanger 800.

Optionally, a same continuous sheet of pollen adsorbing matrix 902 is supported by a plurality of arms 204A-204C in common (that is, 2, 3, 4 or more of such arms), e.g., with each arm 204A-204C movable to some degree independently for reduction of force on plant parts. A range of motion is the potentially limited by an amount of slack material between each arm which may be selected appropriately. Optionally, transport of pollen adsorbing matrix 902 to refresh plant-engaging surface 800A occurs in steps large enough to refresh all arms at once. Optionally, areas of slack convert to areas of pollen adsorbing matrix 902 positioned as plant-engaging surface 800A, and vice versa (e.g. advancing just one “arm's height” at a time), until a long section of pollen adsorbing matrix 902 has been exposed, and then the whole section is transported away and replaced with a fresh length of pollen adsorbing matrix 902.

Spar 911 is referred to alternatively and equivalently as “support girder”, and it is to be understood than any suitable number of support girders, guys, fairings, and sensors are provided per mechanical arm 904.

Reference is now made to FIG. 10, which schematically represents interaction of an artificial pollination system 200 with a plant 1001, according to some examples of the presently disclosed subject matter.

Pollen exchanger 800 is represented at the distal end of mechanical arm 904, with mechanical arm 904A representing mechanical arm 904 in a raked position. Carriage 801 is also represented, as well as housing 930, which may be understood as an indication that various control and sensing components described in relation to FIG. 9D are present, although again, not necessarily in housing 930 itself.

Plant interaction zone 1002 represents a region through which pollen exchanger 800 sweeps as artificial pollination system 200 is drawn along a path indicated by arrow 1005. Optionally, other zones, e.g., portions of zone 1002A more generally, are reached by driving artificial pollination system 200 along other paths, e.g., along a row extending on an opposite side of plant 1001, or in a circle around plant 1001. Branches 1003 are represented schematically.

The size of plant interaction zone 1002 is selected by an operator, typically to include the volume in which inflorescences 501 are densest. Preferably, it is avoided to go so far toward the trunk 1004 of plant 1001 that branches become so stiff that mechanical arm 904 must be refused (given a deliberate raking angle) almost continuously. However where plant rows are narrowly arranged, near-continuous refusal of 904 to a raking angle significantly behind the forward-biased position may be appropriate. Movement through plant interaction zone 1002 involves compromises between bending of plant parts, and active and/or passive refusal of mechanical arm 904 to avoid damaging plant parts by bending them too sharply.

For example, during periods when mechanical arm 904 engages with a particularly stiff branch of a plant, it may be drawn back into a position like that of 904A. This may allow the branch to bend less, and/or at a more distal and flexible section before it snaps past the mechanical arm 904.

In some examples, sensing and active control is used to help maintain an appropriate balance of forces, for example as explained in relation to FIG. 11. It should be understood that additionally or alternatively to adjusting the raking angle around a vertically oriented axis of the support structure (e.g., mast 202), the raking angle is optionally adjusted around a horizontal axis; e.g., by rotating an average plane of a plant-engaging surface 800A of pollen exchanger 800 to a more horizontal orientation. Optionally, a combination of active (controller-maintained) and passive (e.g., spring mechanism maintained) angle control mechanisms is provided; either combined for use around a single axis of rotation, or different for different axes of rotation. For example, to help reduce sudden peak loads, a portion of spar 911 is optionally flexible, and/or reversibly breaking from a spring biased forward configuration to a bent configuration (that is, jointed at the breaking region). Optionally, the breaking mechanism is tuned to bend at a force which is about equal to or higher than a threshold that active control is set to normally maintain. Optionally, such reversible breaking and/or bending is coupled to sensing (e.g., strain, contact, and/or proximity sensing) which is used as an indication of plant force by controller 934, e.g., as part of the operations of controller 934 described in relation to FIG. 11.

Reference is now made to FIG. 11, which is a block diagram schematically representing components, inputs, and other data structures of a controller 934 for a powered mechanical arm 904, according to some examples of the presently disclosed subject matter.

Controller 934 optionally corresponds to controller 934 of e.g., FIG. 10D. Its functionally defined components include memory 1101, and processors 1109, although these functions may be implemented in an integrated fashion, e.g., as an ASIC or other form of processing circuitry. Communication interface 1141 optionally implements any suitable form of wired and/or wireless device communication, e.g., WiFi, Zigbee, Bluetooth, and/or electromagnetic (e.g., radio and/or optical wavelength) and/or acoustic (e.g., ultrasound) signaling more generally. Optionally, Communication interface 1141 is used to communicate with a remote user interface 144 (e.g., on a cell phone or other portable device), and/or to receive data, for example, data as described in relation to any of blocks 1140, 1110, and/or 1120. Optionally, communication interface 1141 is used in communicating one or more of motor control commands 1130 and/or transport motor control commands 1150.

Optionally, memory 1101 receives sensor inputs 1110 from one or more sensors 936, 936B, the sensors being positioned on mechanical arm 904, artificial pollination system 200, carrier 801, tractor 840, and/or within the field or orchard being farmed as appropriate. Sensors are configured to sense present positioning of mechanical arm 904, present forces acting on mechanical arm 904, and/or to assist in predicting positioning and/or forces, e.g., based on geographic information about the field or orchard, and/or by synchronizing with events previously recorded while travelling in the same area of the field orchard. It should be understood that although sensor inputs are optionally provided directly to processor 1109 itself, e.g., as register entries, such cases should still be considered as using elements of memory 1101, as functionally defined.

Examples of sensor inputs 1110 include system attitude(s) 1104 (i.e., angle with respect to gravity of one or more parts of the system, such as mast 202), arm angle 1105 (e.g., swing angle), motor force 1106, geographical position 1107, inertial state 1108, and images 1108B. Inertial state 1108 optionally comprises any one or more of velocity and acceleration, optionally in any direction of translation or rotation. System attitude 1104 may be a subset of inertial state 1108, i.e., a measurement of the direction of acceleration due to gravity. Sensing and/or sensor inputs described in relation to FIG. 14 are optionally provided as appropriate; e.g., sensing of electrical state 1108C.

Sensors which produce these sensor inputs (also referred to herein as “indications”) may be of any type suitable for the indication mentioned, e.g., one or more motor encoders, Hall effect sensors, MEMS (micro electromechanical sensors, e.g., of acceleration, rotation, and/or magnetic field strength/orientation), GPS sensors, and/or another type of sensor. Images 1108B are optionally obtained using a LIDAR device, an optical camera, or another type of imaging sensor.

In some examples, memory 1101 stores an arm representation 1120 used and/or maintained by processor 1109 to determine commands including arm motor control commands 1130 (communicated, e.g., to motor 932 and/or a controller thereof), and/or transport motor control commands 1150 (communicated, e.g., to motors 926 of the matrix transport 933, and/or a controller thereof).

In particular, arm model 1103 comprises a data representation of mechanical arm 904 suitable for algorithmic interpretation and/or manipulation, such as its dimensions, mass distribution, and mounting angle. Optionally, features of the kinematics of mechanical arm 904 are represented such as flexible and/or jointed regions. Positioning of the plant-engaging surface 800A of pollen exchanger 800 may be modeled; e.g., the position of the portion of pollen adsorbing matrix 902 which is exposed to contacts with plants resulting in pollen exchange (e.g., pollen collection and/or pollinating).

Arm state 1102 optionally represents the current state of mechanical arm 904, e.g., the state of parameters of arm model 1103, and/or states such as current translational and/or angular orientation and velocity in one or more dimensions.

Optionally, parameters of the pollen collecting and/or pollination task are represented by block 1140, e.g., location(s) of plants which are to be engaged with by a plant-engaging surface 800A of the pollen exchanger, a total length of travel, and optionally shapes of plants and/or shapes/locations of targeted plant interaction zones 1002. Optionally, the data structures of block 1140 represent features of the farm or orchard itself, e.g., locations of pathways, locations of obstacles, measurements of pathway evenness as obtained, e.g., from previous operations, or other data characterizing the terrain along which pollen exchange operations are occurring.

Processor 1109 implements one or more algorithms, by means of which the inputs and other data structures of memory 1101 are converted to commands 1130 (e.g., controlling positions of mechanical arms 904) and/or commands 1150 (e.g., controlling transport of pollen adsorbing matrix 902). Non-limiting examples of considerations which are optionally taken into account by these algorithms are next described.

In general (but not necessarily), processor 1109 may be configured with the target of isolating displacement forces on mechanical arm 904 due to plant engagement from displacement forces due to other causes, such as uneven terrain, travel accelerations, and inherent design. Remaining forces are optionally attributed to plant engagement, and targets for maximum forces exerted on plants applied accordingly. This potentially enables effective targeting of restorative forces that remain within limits set to avoid damaging plants with which artificial pollination system 200 is engaged by contact.

In an example: a mechanical arm 904 is presently held at a forward-biased target location by commands which result in a 0.5 A (half Ampere) electrical current through motor 932. The motor is expected, according to system design and/or status information, to exert an additional 4 N of force at its tip when the electrical current reaches 0.7 A. Accordingly, forces tending to displace mechanical arm 904 are resisted fully up to 0.7 A, but above this level, processor 1109 limits (and optionally reduces) motor power, resulting in mechanical arm 904 being pushed back to a more raked state. Upon release of plant interaction forces, processor 1109 may attempt to advance mechanical arm 904 back again towards its forward-biased state.

In some examples, mechanical arm 904 is mounted to its support structure in such a way that it hangs to a particular equilibrium angle, when artificial pollination system 200 is otherwise positioned level and at equilibrium. For example, it may “droop” slightly, although a substantially level installation without a specific equilibrium angle is not excluded. Where the equilibrium angle is displaced from the targeted forward-biased angle of mechanical arm 904, a motor 932 is optionally operated with sufficient torque and/or power to return it to position against the gradient of gravity. Additionally or alternatively, the force needed to do this is optionally discounted from calculations of plant interaction forces.

The equilibrium angle and amount of “droop” may be known by design, and/or it may be calibrated for, e.g., by sensing (e.g., using an encoder sensor 936) the equilibrium angle. Optionally, the equilibrium angle is not known, and controller logic simply operates to draw mechanical arm 904 toward its targeted position. However, knowing the equilibrium angle and the forces required to overcome it are a potential advantage is distinguishing forces exerted on mechanical arm 904 merely by gravity, and those exerted from plants which mechanical arm 904 encounters.

During travel, carrier 801 may be reasonably expected to encounter uneven ground, resulting in the support structure of mechanical arm 904 experiencing tilt. Changes in some components of tilt may be relatively slow and continuous (e.g., in travelling over hilly ground). Some components of tilt may change more rapidly, e.g., due to uneven conditions of the path surface over which carrier 801 is travelling such as holes and mounds. In some examples, processor 1109 receives continuously updated indications of tilt, e.g., mast attitude 1104. Optionally, power delivered to motor 932 is adjusted to counteract tilt acceleration of mechanical arm 904, preferably before changing tilt acceleration imparts significant angular momentum. Additionally or alternatively: travel accelerations of carrier 801 (e.g., starting, stopping, and turning) potentially impart swing forces on mechanical arm 904. In some examples, processor 1109 monitors travel accelerations, and compensates appropriately by commands provided to motor 932.

In some examples, for which terrain is well-known in advance, processor 1109 optionally uses terrain data (e.g., from the data of block 1140) to at least partially anticipate tilt changes. This potentially allows reducing a peak torque requirement on motor 932, by allowing it to produce movements of mechanical arm 904 optionally in advance of the tilt which they counteract.

In cases where forces impart momentum (velocity) to mechanical arm 904, processor 1109 optionally operates to damp this momentum using servo control techniques. Optionally, arm state 1102 represents, e.g., angular arm velocity and/or acceleration (e.g., as determined from periodic measurements of arm angle 1105), while arm model 1103 represents how mechanical arm 904 responds to motor control commands 1130 while having this state.

It should be understood that measurements of forces and/or use of these measurements are optionally omitted and/or disregarded, where it is determined that they are likely to be small enough and/or infrequent enough that damage to plants is unlikely and/or at an acceptable level. For example, it may be determined that forces due to inherent arm droop and/or device shaking during travel can be disregarded.

It should be understood that settings are optionally substituted for measurements as such in certain examples, and/or in certain circumstances. For example, an operator may be tasked with manually setting the targeted forward-bias position of mechanical arm 904 according to the slope of the terrain and/or according to other factors such as row width. The processor may learn from this setting which motor control output command (e.g., targeted power output) should be considered as “equilibrium”, and exert additional restorative forces accordingly, without necessarily measuring and accounting for accelerations explicitly. Optionally, nevertheless, such an operator setting is accounted for along with one or more measurements indicative of forces applied to the mechanical arm 904 separate from plant-exerted forces.

Optionally, indications of excessive force (e.g., a sufficiently large divergence between commanded motor forces and resulting angle of mechanical arm 904, whatever the cause) are treated as an absolute indication of a problematic interference condition. Processor 1109 optionally responds, for example, by refusing (retracting) mechanical arm 904 to a more steeply raked angle (e.g., in an angle that “protects” it), and/or by abandoning control attempts until operator input is given which permits proceeding.

Optionally, in examples having a plurality of mechanical arms 904, forces due to branch interference are estimated at least in part by distinguishing commonalities among forces experienced by different arms from differences. For example, forces indicated by events seen only in one arm are attributed to “plant interference”, while forces indicated by events seen in common for each arm are attributed to non-plant causes such as ground slope and/or vehicle acceleration.

Optionally, periods of relative quiet between encounters with plants and/or near the beginning/end of such encounters are used to renew a baseline state representation of the accelerations experienced by mechanical arm 904, with appropriate maximum forces exerted by compensatory commands being calculated during the peak of the plant encounter relative to this baseline state.

Optionally, images 1108B are used as indications which help distinguish forces due to plant contacts. For example, periods with plant contact and without plant contact are distinguished according to whether or not plants are imaged in contact with mechanical arms 904. Changes in force measured by other sensors (e.g., arm angle 1105, motor force 1106, or another measurement) are then attributed to plant contacts as appropriate, or else assigned other aspects of the system state, and this apportionment then used as a basis for determining what motor control commands 1130 should be issued to reduce plant damage, or for another purpose.

In some examples, transport motor control commands 1150 are calibrated to help match the use of surface area of pollen adsorbing matrix 902 to the progress of a pollen collecting and/or pollen distributing operation. For example, advances of pollen adsorbing matrix 902 are performed at a rate consistent with using the whole available surface area not before and/or by the end of a row targeted for pollen exchange operations. Optionally, the advance is performed per unit of distance traveled. Optionally advance is per plant visited, e.g., pollen adsorbing matrix 902 is advanced by one “frame” for each estimated plant visited.

In some examples, transport motor control commands 1150 are modulated according to plant contacts. For example, during periods in which plant contact is detected (e.g., by force measurements and calculations, by imaging, and/or by another method), transport of pollen adsorbing matrix 902 is optionally actuated to stimulate pollen transfer, e.g., by oscillation of pollen adsorbing matrix 902 or another movement. Optionally, a shaking (e.g., vibratory) mechanism is provided which induces shaking of pollen adsorbing matrix 902 preferentially during a period of plant contact (e.g., as determined from force indications, imaging, fluctuations in electrostatic field potential, or another indication).

Optionally, periods of low plant contact force are used as an indication of movement to a new plant, and pollen adsorbing matrix 902 shifted accordingly to refresh all or a part of plant-engaging surface 800A.

Reference is now made to FIGS. 12A-12B, which schematically represent movement of a pollen adsorbing matrix 902 by a matrix transport 933 of a powered mechanical arm 904, according to some examples of the presently disclosed subject matter. Components as labeled are as described, e.g., in relation to FIGS. 9A-9E. Arrows 1201 of FIG. 12A indicate a direction of travel of pollen adsorbing matrix 902, e.g., as it is being wound from roller 920A to roller 920B, for example, during operations to collect pollen. Arrows 1202 of FIG. 12B indicate a reversed direction of travel of pollen adsorbing matrix 902, e.g., as it is being wound from roller 920B to roller 920A, for example, during operations to pollinate. It should be understood that winding direction may be in any direction, and that winding is optionally not reversed between operations, but rather a rewind stage occurs after pollen collection, with pollination occurring in the same order of exposure as was used for pollen collecting.

Optionally, winding advance is continuous during pollen collection, e.g., as a function of time and/or distance, optionally calibrated for an anticipated duration and/or distance over which pollen transfer operations occur. Optionally, winding advance occurs in discrete jumps, e.g., by time or distance, and/or per plant visited. Optionally, jumps in winding advance are enough to present a new “clean” frame to each successive plant visited. Optionally, the jumps are smaller, e.g., half a frame, a third of a frame, or another value (with a full frame being roughly equivalent to the area of pollen adsorbing matrix 902 which is fully exposed at one time.

Optionally, winding repeatedly advances (e.g., as shown in FIG. 12A) and reverses (e.g., according to arrows 1202 of FIG. 12B) during a period of pollen collection and/or pollination. This provides a potential advantage of providing additional mechanical agitation and/or surface area exposure to free pollen from and/or transfer pollen to target plant(s). The reversal period can be restricted to about the size of a single frame (e.g., half a frame up to about double a frame size), and/or reversal can occur when one of the ends of pollen adsorbing matrix 902 is reached.

It is a potential advantage to maintain a record of which parts of pollen adsorbing matrix 902 were exposed to pollen collecting at which region of the farm/orchard and/or for which specific plants. This potentially allows plant-to-plant matching of pollinator plan to pollinated plant. Optionally, pollination on different days and/or different pollination passes down a row of plants is deliberately adjusted to ensure that a repeatedly pollinated plant receives pollination from more than one plant. Optionally, records of pollination success (e.g., per-tree productivity records) are indexed to pollinating plants, and the records used to adjust pollination plans during subsequent growing seasons.

Potentially, there may be pollen transfer occurring between layers of a rolled up pollen adsorbing matrix 902, e.g., from front side to back side. To potentially mitigate this, one side of pollen adsorbing matrix 902 may be made smooth, or otherwise adjusted to reduces its adherence. Optionally, the front and/or back of pollen adsorbing matrix 902 is scraped (e.g., by a scraper), and collected pollen transported to be re-adhered to a surface of pollen adsorbing matrix 902 for exposure to plants being pollinated.

In some examples, a portable handheld pollen collector device 100, and/or pollinating device 50 is provided with a matrix transport 933, for example, a belt- or scrolling-type matrix transport 933 coupled to pollen adsorbing matrix 12. Although potentially increasing weight, this provides potential advantages for inducing automatic motion of pollen adsorbing matrix 12 to assist pollen exchange.

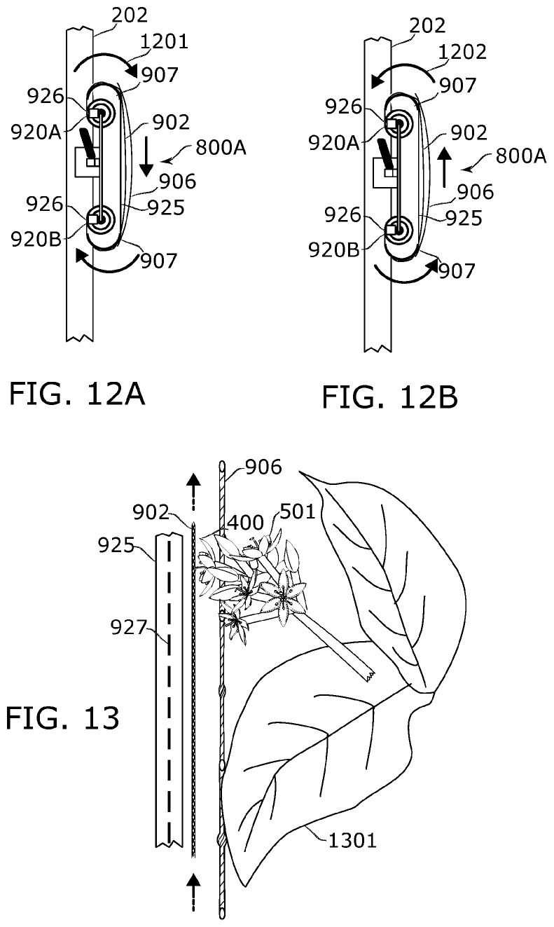

Reference is now made to FIG. 13, which schematically represents a portion of an inflorescence 501 protruding through a protective net 906 into proximity with a portion of a pollen adsorbing matrix 902, according to some examples of the presently disclosed subject matter.

Protective net 906 and pollen adsorbing matrix 902 are shown partially and in cross-section. Also illustrated is a cross-section through pollen attractor 925, including cross-sections of spirally arranged coil 927. Protective net 906, in some examples, has apertures large enough to admit flowers 400 on inflorescences 501, but small enough to exclude larger features such as leaves 1301.

Protective net 906 is optionally provided to any example of a pollen collecting and/or pollinating device herein, e.g., whether hand-held or not (e.g., as in FIGS. 1-3C), and whether provided with a static pollen adsorbing matrix 222 (e.g., as in FIG. 8A-8B) or a transporting pollen adsorbing matrix 902 (e.g., as in FIGS. 9A-9E).

It should be understood that protective net 906 does not necessarily prevent flower contact; rather it acts to resist penetration of and/or to pollen adsorbing matrix 902 by larger plant parts, e.g., leaves (in particular), and potentially branches, twigs, and/or thorns. Use of protective net 906 is optional. However, (e.g., for use during collection of pollen in particular), protective net 906 is optionally provided with gaps smaller even than flowers 400, since pollen 401 is being attracted electrostatically in any case.

Protective net 906 provides a potential advantage for reducing losses of adsorbed pollen on pollen adsorbing matrix 902 to encounters with foliage, during pollen collecting and/or during pollination.

For pollination to occur, stamens 410 of flowers 400 may reach contact with pollen adsorbing matrix 902 and pollen grains it carries. It is not excluded that pollen 401 may be brushed loose from pollen adsorbing matrix 902 by another plant part, allowing it to reach stigma 409 through the air. It may be a potential advantage, however, if such plant parts are at least more likely to be located near receptive stigmas 409.

For pollen collection operations, proximity without contact may be sufficient to allow a high voltage potential applied via spirally arranged coil 927 (e.g., of 1 kV or more, for example, as described in relation to pollen collector device 100) to attract pollen towards it, which becomes adsorbed on pollen adsorbing matrix 902 as a result.

Vibrations due to repeating plant contacts with protective net 906 as it travels past a plant potentially also assist in dislodging pollen, and this effect may carry through from plant parts in contact with protective net 906 to flowers relatively distant from pollen adsorbing matrix 902 and/or protective net 906. This has a potential advantage for improving pollen collection efficiency.

Optionally, protective net 906 is provided with a plurality of different aperture sizes, allowing a plurality of vibration frequencies to be applied through plant parts which strike against cords of protective net 906. Potentially this increases a likelihood of exposing anthers 407 to a vibration frequency which encourages them to dehisce (e.g., a resonant frequency which happens to induce larger amplitude shaking, and/or to which a pistil 408 is particularly susceptible). For example, protective net 906 is optionally provided with gaps ranging over a factor of 2, 3, 4, 5 or more in size. Optionally, gaps in protective net 906 are set to have widths in the direction of travel in the range of about 0.5 cm, 1 cm, 2 cm, 3 cm, 4 cm, 5 cm, or another size. It should be noted, furthermore, that diagonally oriented apertures naturally create zones where some pairs of contacts happen in relatively short mutual succession, and other pairs of contacts happen in relatively long mutual succession (e.g., depending on how close to the corner of an aperture contacts take place).

It should be understood that protective net 906 is an example of a size-selective barrier more generally; the size-selective barrier being configured with gaps large enough to admit at least one of pollen and flower parts beyond it, but small enough to prevent a majority of foliage contacts from penetrating. Optionally, examples described as being provided with a protective net 906 are provided with another type of size-selective barrier. For example, the size-selective barrier may be implemented with barrier strands comprising bars (e.g., of wire and/or polymer), bands (e.g., of metal and/or an elastic polymer), and/or string/rope.

Optionally, the barrier strands are crossing or non-crossing, and optionally include sets of parallel barrier strands. Sets of non-crossing barrier strands are optionally arranged with slight angular differences, which potentially results in gradients of contact frequencies over the extent of plant-engaging surface 800A. Optionally, size-selective barriers are tuned (that is, adjusted in the spacing, density, and/or angles of the barrier strands), e.g., according to observed effectiveness in increasing pollen yield and/or pollination effectiveness, e.g., effectiveness due to less contact with foliage, different vibration-inducing properties, or for another cause, known or unknown. Optionally tuning is accomplished by stretching a protective net 906 or other size-selective barrier relatively more or less, e.g., along a direction of travel. Optionally barrier strands are added or removed to accomplish tuning.

Optionally, spacing between protective net 906 and pollen adsorbing matrix 902 is selected and/or adjustable to assist in excluding foliage, e.g., increased during pollen collection, to help ensure that more slight penetrations of protective net 906 still do not reach pollen adsorbing matrix 902.

In the case of hand-operated pollen collector devices 100, protective net 906 (or another size-selective barrier) is optionally provided; for example, provided on its own swing-away and/or lift-away frame. Optionally, the size-selective barrier is pulled over charged pollen attractor 110 (e.g., stretched and pulled over), once a pollination cartridge 10 has been placed on it, and pulled off again to change pollination cartridge 10. Optionally, each pollination cartridge 10 includes its own protective net 906 or other size-selective barrier. Optionally protective net 906 is set with apertures sized differently in different directions (e.g., by a factor of 2 or more), allowing device operators to accomplish different shake frequencies according to which direction they move pollen collector device 100 while it is in contact with flowers 400 and/or inflorescences 501.

### GENERAL

As used herein with reference to quantity or value, the term “about” means “within =10% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the present disclosure may include a plurality of “optional” features except insofar as such features conflict.

Throughout this application, embodiments may be presented with reference to a range format. It should be understood that the description in range format is merely for convenience and brevity and should not be construed as an inflexible limitation on the scope of descriptions of the present disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although descriptions of the present disclosure are provided in conjunction with specific embodiments, it is evident that many alternatives, modifications and variations will be apparent to those skilled in the art. Accordingly, it is intended to embrace all such alternatives, modifications and variations that fall within the spirit and broad scope of the appended claims.

It is appreciated that certain features which are, for clarity, described in the present disclosure in the context of separate embodiments, may also be provided in combination in a single embodiment. Conversely, various features, which are, for brevity, described in the context of a single embodiment, may also be provided separately or in any suitable subcombination or as suitable in any other described embodiment of the present disclosure. Certain features described in the context of various embodiments are not to be considered essential features of those embodiments, unless the embodiment is inoperative without those elements.

It is the intent of the applicant(s) that all publications, patents and patent applications referred to in this specification are to be incorporated in their entirety by reference into the specification, to the same extent as if each individual publication, patent or patent application was specifically and individually noted when referenced that it is to be incorporated herein by reference. In addition, citation or identification of any reference in this application shall not be construed as an admission that such reference is available as prior art to the present disclosure. To the extent that section headings are used, they should not be construed as necessarily limiting. In addition, any priority document(s) of this application is/are hereby incorporated herein by reference in its/their entirety.

1-29. (canceled)

30. A pollination system mountable on a carrier configured to carry the system along a pollination path alongside plants to enable the system to assist in pollination of the plants, the system comprising:
a support structure;
at least one mechanical arm, attached to the support structure, and positioning a plant-engaging surface of a pollen exchanger where the plant-engaging surface engages in contacts with the plants as the system moves along the pollination path;
a motor, attached to the at least one mechanical arm, and operable to change at least an angle at which the plant-engaging surface contacts the plants; and
a controller, configured to operate the motor to adjust the angle in response to one or more indications of force exerted upon the plants by the contacts.

31. The pollination system of claim 30, wherein the one or more indications of force include a sensed indication of a change in the angle produced by the contacts with the plants.

32. The pollination system of claim 30, wherein the one or more indications of force include an indication of a change in how force exerted through the motor affects the angle produced by the contacts with the plants.

33. The pollination system of claim 30, wherein the controller operates the motor to reduce force of plant contact in response to the one or more indications of force.

34. The pollination system of claim 30, wherein the controller responds to indications of force of plant contact at least greater than 15 N by movement of the mechanical arm to an increased raking angle.

35. (canceled)

36. The pollination system of claim 34, wherein the controller initiates the movement to the increased raking angle by powering the motor to refuse the arm in a direction opposite a direction of travel of the system along the pollination path.

37. The pollination system of claim 34, wherein the controller initiates the movement to the increased raking angle by reducing power to the motor, allowing the arm to swing in a direction opposite a direction of travel of the system along the pollination path.

38. The pollination system of claim 30, wherein the one or more indications of force combine indications of force exerted on the mechanical arm by plants, and indications of force exerted on the mechanical arm from one or more additional sources.

39. The pollination system of claim 38, wherein the one or more additional sources comprise at least one of a tilt angle of the system and an inertial state of the system, with measurements thereof provided among the indications of force.

40. The pollination system of claim 38, wherein the controller estimates force exerted on the mechanical arm by plants by subtracting estimated force exerted on the mechanical arm by the one or more additional sources from a total force estimated from the one or more indications of force.

41. The pollination system of claim 30, wherein the controller comprises a memory and processor, wherein the memory stores instructions which instruct the processor to:
access sensor inputs indicative of forces exerted on the at least one mechanical arm;
estimate a force exerted on the at least one mechanical arm by plants, using the sensor inputs; and
initiate operation of the motor to adjust the angle of the arm, according to the estimated force.

42. The pollination system of claim 41, wherein the controller initiates operation of the motor to decrease the estimated force exerted on the mechanical arm by plants, according to a comparison of the estimated force with a threshold force.

43. The pollination system of claim 42, wherein the threshold force is less than about 15 N.

44. The pollination system of claim 30, wherein the adjusted angle is an angle around an axis of rotation which is substantially vertical.

45. The pollination system of claim 30, wherein the adjusted angle is an angle around an axis of rotation which is substantially horizontal.

46. The pollination system of claim 30, wherein the pollen exchanger comprises an electrically powered pollen attractor comprising an insulated electrode which generates an electrostatic field upon receiving high voltage.

47. The pollination system of claim 46, comprising a pollen adsorption matrix extending across the pollen attractor and providing the plant-engaging surface, wherein the electrostatic field attracts pollen to the pollen adsorption matrix.

48. The pollination system of claim 47, comprising a size-selective barrier extending across the plant-engaging surface, and positioned to restrict sizes of plant parts which contact the pollen adsorption matrix.

49. The pollination system of claim 30, wherein the mechanical arm includes a flexible or jointed region which is biased to hold a portion of the mechanical arm in a more forward position, but bend under a sufficient force to relieve plant contact forces.

50. The pollination system of claim 30, wherein the controller operates the motor to adjust the angle to a default angle, unless the one or more indications indicate that this would exert an excessive force upon plant parts contact the at least one mechanical arm.

51. The pollination system of claim 50, wherein the default angle is about perpendicular to the pollination path.

52-234. (canceled)
